(19) United States
(12) Patent Application Publication
(10) Pub. No.: US 2026/0297534 A1
(43) Pub. Date: Oct. 1, 2026

(54) CANCER MODELS

(71) Applicant: CARCINOTECH LTD, Midlothian (GB)

(72) Inventors: Ishani MALHOTRA, Midlothian (GB); Kamila PAWLICKA, Midlothian (GB)

(21) Appl. No.: 19/480,387

(22) PCT Filed: May 3, 2024
(86) PCT No.: PCT/GB2024/051173

§ 371 Date: Oct. 31, 2025

(30) Foreign Application Priority Data
May 3, 2023 (GB) 2306535.2

(51) Int. Cl. C12N 5/09

(52) U.S. Cl. CPC C12N 5/0693; C12N 2502/1107; C12N 2502/1114; C12N 2502/1121; C12N 2502/1157; C12N 2502/1164; C12N 2503/02; C12N 2513/00; C12N 2533/90

(57) ABSTRACT

Disclosed are tissue and/or tumour models or constructs that are able to mimic an in vivo environment, such a cancer or diseased tissue microenvironment. The disclosure also extends to methods of making such models or constructs, kits for making the described models or constructs, cell culture media and uses of the media, models or constructs.

### FIELD

The present disclosure relates to models or constructs that are able to mimic an in vivo environment, such a cancer microenvironment. The disclosure also extends to methods of making such models or constructs, kits for making the described models or constructs, cell culture media and uses of the media, models or constructs.

### BACKGROUND

Bioengineered constructs or artificial models are of increasing importance in the development of new therapies or drugs and offer the opportunity to reduce the reliance on animal models. However, it is not trivial to provide a construct or model that accurately mimics an in vivo environment. This is particularly true for a tumour microenvironment, which is highly complex. Many cancer models involve 2D and/or 3D cell cultures in conventional plastic dishes or even on a gel matrix. More recent studies may also involve the use of scaffolds to study 3D structures of cancers and their interactions with other cells. A recent publication (WO 2020/254660 A1) provides a specialised microfluidic device for mimicking a cancer microenvironment. In addition, Hermida et al. (“Three dimensional in vitro models of cancer: (bio)printing multilineage glioblastoma models”; Advances in Biological Regulation 75 (2020), 100658) and Tang et al. (“Three dimensional bioprinted glioblastoma microenvironments model cellular dependencies and immune interactions”; Cell Research (2020) 30: 833-853) describe bioprinted models of glioblastoma using multiple cell types.

However, there remains a need to provide artificial constructs or models that can mimic a real-life cancer microenvironment, which recapitulates the characteristics of a patient's tumour microenvironment, that can mimic a cancer heterogeneity, and/or that can be made quickly and/or cost effectively.

### SUMMARY

The present disclosure provides novel media for maintaining and/or expanding cells derived from patient samples—for example tissue samples and/or tissue biopsies—including tumour samples/biopsies. From these samples, the methods described herein may provide a dissociated cell mix which is characterised (at least in terms of cellular content) and then subject to a maintenance and/or expansion protocol which exploits any of the media described herein. The maintenance and/or expansion protocol will yield an expanded population of cells which can be bio-printed. The inventors have discovered that the media provided here help reduce the length of any maintenance or expansion period and that it helps preserve the cellular characteristics of the original sample. This ensures that any constructs (bio)printed from cells maintained and expanded using a media of this invention, represent an accurate (in terms of cell composition and/or organisation) copy of the original sample. Such accurate (bio)printed constructs are very useful for drug testing assays and the like.

It should be noted that the terms “comprise”, “comprising” and/or “comprises” is/are used to denote that aspects and embodiments of this invention “comprise” a particular feature or features. It should be understood that this/these terms may also encompass aspects and/or embodiments which “consist essentially of” or “consist of” the relevant feature or features.

Accordingly, in some examples, the method may consist of (or consist essentially of) the steps provided above.

According to an aspect of the disclosure, there is provided a method of making a tissue construct or a tumour construct, said method comprising:
(a) determining the cellular profile of a tissue or tumour biopsy obtained from or provided by a subject;
(b) preparing a cell mixture to mimic the determined cellular profile of the tissue or tumour biopsy;
(c) optionally supplementing said cell mixture with immune cells derived or obtained from, or provided by, said subject; and
(d) using said (optionally supplemented) cell mixture to prepare a tissue or tumour construct.

The method described herein may comprise the additional step of dissociating a biopsy (for example a tissue biopsy or a tumour biopsy) to prepare a dissociated cell mix. The dissociated cell mix may then be maintained and/or expanded in the presence of any of the media (e.g. the expansion media) described herein. After a period of time (which period of time is shorter than if the maintenance/expansion step had been conducted using a prior art medium) the total number of cells will have expanded to a number which is suitable for the (bio)printing of a tissue or tumour construct according to this disclosure.

It should also be noted that the optional ‘supplementing’ step may be needed should any expanded cell population not comprise the required number of cells and/or lack one or more specific cell types present in the original sample. This may occur where the cells dissociate from the sample grow and expand more slowly than expected.

It should be noted that while this invention will now be described with reference to tumour biopsies and the (bio)printing of a tumour construct, the various methods, protocols and techniques described herein may equally apply to the (bio)printing of other tissue/structure types.

Accordingly, in one embodiment the disclosure provides a method of making a tissue construct, said method comprising:
(a) determining the cellular profile of a tissue sample obtained from or provided by a subject;
(b) dissociating the tissue sample to provide dissociated cells;
(c) expanding the dissociated cells in the presence of any of the media (e.g. expansion media) described herein; and
(d) using the expanded dissociated cells to prepare a tissue construct.

In a further teaching, the disclosure provides a method of making a tumour construct, said method comprising:
(a) determining the cellular profile of a tumour biopsy or sample obtained from or provided by a subject;
(b) dissociating the biopsy or sample to provide dissociated cells;
(c) expanding the dissociated cells of the biopsy sample in the presence of any of the media (e.g. expansion media) described herein; and
(d) using the expanded dissociated cells to prepare a tumour construct.

The disclosure further extends to tissue or tumour constructs obtainable by either of the abovementioned methods. It should be noted that a tissue or tumour construct made by these methods is characterised by the following features: (i) it retains a cellular composition which is substantially identical to the cellular composition of the original sample; (ii) it retains an immune cell profile which mimics (or is substantially identical or similar to) the immune cell profile of the tumour biopsy, (iii) as compared to the cellular organisation of the original sample, it has a substantially similar or identical cellular organisation and/or (iv) is genetically similar to the genetic profile of the original sample (e.g. tissue or tumour biopsy). The terms ‘substantially similar’ or ‘substantially identical’ may mean that the resulting construct comprises at least one cell representative of each cell type present in the original sample. Most usually, the resulting construct comprises cells of each cell type present in the original sample at a number representative of the number of those cells present in that sample. The term “genetically similar” may mean that the level of gene expression and/or number of genes expressed in a (bio)printed construct of this disclosure (for example a (bio)printed tissue or tumour construct) is at least 60%, 65%, 70%, 75%, 80%, 85%, 90%, 91%, 92%, 93%, 94%, 95%, 96%, 97%, 98%, 99% or 100% of the gene expression of number of genes expressed in the original sample (e.g. the tissue or tumour biopsy).

The term ‘biopsy’ may embrace any sample or section of a tumour. As such, the terms ‘biopsy’ and ‘sample’ may be interchangeable. A method of this disclosure may use a plurality of biopsies all taken from the same tumour. The biopsies may be taken from different sites within the same tumour. Comparing the cell profiles of a plurality (e.g. two or more) biopsies from the same tumour may allow a more representative cell profile to be determined.

The constructs of the present disclosure are in vitro microenvironments or artificial models or constructs that have the capacity to mimic an in vivo microenvironment. In particular, the tumour constructs of the present disclosure are capable of mimicking an in vivo tumour microenvironment of the subject from whom the tumour biopsy was obtained or provided.

The constructs and methods of the present disclosure may provide an artificial model and/or have the capacity to mimic the in vivo microenvironment of any number of different cancers. Indeed, the type of cancer that is being mimicked is dependent upon and/or determined by the nature of the tumour biopsy obtained from or provided by the subject. By way of example, the tumour construct may provide an artificial model and/or have the capacity to mimic an in vivo microenvironment of a cancer selected from the following non-limiting examples, a brain tumour (such as glioblastoma multiforme (GBM)), breast cancer, lung cancer, colorectal cancer, ovarian cancer, prostate cancer, head and neck cancer, pancreatic cancers, rare cancers and paediatric cancers.

Without wishing to be bound by theory, the present inventors have unexpectedly identified that the tumour constructs made by the methods disclosed herein recapitulate the immune cell composition of the in vivo tumour microenvironment and importantly, the immune cell composition of the construct is maintained over time. Further, the constructs disclosed herein were unexpectedly found to maintain the cellular profile or characteristics of the primary tumour from which the biopsy was obtained. The constructs of the present disclosure overcome some of the challenges of existing tumour models, which include, but are not limited to, maintaining the viability, function and/or the heterogeneous composition of the immune cells over an extended period of time. Moreover, the composition and/or structural organisation of the tumour construct substantially replicate that of the tumour biopsy by using immune cells derived or obtained from, or provided by, the same subject or patient (sometimes referred to herein as “matched immune cells”).

Consequently, the constructs described herein or the constructs made by the methods disclosed herein may be used to provide an indication or determine how the subject may respond to a treatment or combination of treatments with improved specificity. The disclosed constructs may facilitate screening of test agents in models that more closely reflect the tumour characteristics of a particular patient and/or provide a more accurate prediction of a clinical response in a particular patient, for example.

It will be appreciated by the skilled addressee that a tumour construct of the present disclosure may also be made by using a cell mixture to make an initial tumour construct then (optionally) supplementing the initial tumour construct with immune cells derived or obtained from, or provided by the same subject or patient.

As such, in another aspect, there is provided a method of making a tumour construct, said method comprising:
(a) determining the cellular profile of a tumour biopsy obtained from or provided by a subject;
(b) preparing a cell mixture to mimic the determined cellular profile of the tumour biopsy;
(c) using said cell mixture to prepare a tumour construct; and
(d) optionally supplementing said tumour construct with immune cells derived from or provided by said subject.
   The step of ‘preparing’ a cell mixture to mimic the determined cellular profile of the tumour biopsy, may comprise dissociating the tumour biopsy to provide dissociated cells and then expanding the dissociated cells using any of the media (e.g. expansion media) described herein. The expanded dissociated cells may then be used (as described herein) to prepare a tumour construct.

In order to recapitulate or mimic the characteristics of the primary tumour (or tissue), the methods of the present disclosure involve determining the cellular profile of a tumour (or tissue) biopsy obtained from or provided by a subject.

As used herein, the phrase “determining the cellular profile of a tumour biopsy” typically refers to assessing the morphological, genotypic and/or phenotypic characteristics of the tumour biopsy. The skilled person would recognise various techniques known in the art which may be employed to determine the morphological, genotypic and/or phenotypic characteristics of the tumour. For example, microscopy, immunohistochemistry and/or immunofluorescence imaging may be performed to characterise the morphological, phenotypic or molecular characteristics (e.g. protein expression) of the tumour biopsy. In addition, or alternatively, one or more sequencing methods may be conducted to determine the genotypic characteristics of the tumour biopsy, such as RNA-seq, whole exome sequencing and/or genome wide sequencing, for example. The sequencing methods may comprise single-cell sequencing method(s) or bulk sequencing method(s), or a combination thereof.

In addition, determining the cellular profile of a tumour biopsy may also encompass determining cell counts and/or proportions of various cells types of the tumour biopsy, such as by using fluorescence activated cell sorting techniques, for example. For instance, the skilled person in the art would be capable of determining the cell number and/or percentage of each cell type present in the tumour biopsy (e.g. using FACS), in order to determine the number and/or percentage of cells that need to be added to the cell mixture and/or tumour construct in order to mimic or substantially replicate the tumour biopsy. By making a tumour model that substantially mimics or recapitulates the heterogeneous cell population found in the tumour biopsy, which includes the heterogeneous immune cell population, it provides a more accurate personalised medicine approach to modelling the in vivo tumour microenvironment.

As used herein, the phrase “determining the cellular profile of a tissue biopsy” typically refers to assessing the morphological, genotypic and/or phenotypic characteristics of the tissue biopsy. This may be carried out using any of the methods and techniques as described above in relation to a tumour biopsy.

In some examples, subsequent to determining the cellular profile of the tissue or tumour biopsy, the proportions or percentages of the various cell types of the tissue or primary tumour as found in the biopsy may be used as a reference when preparing a cell mixture to mimic the determined cellular profile of the tissue or tumour biopsy. In some examples, the proportions or percentages of the various immune cell types of the tissue or tumour as found in the biopsy may be used as a reference when (optionally) preparing the immune cells derived from or provided by the subject to supplement the cell mixture or tissue or tumour construct.

The term “cell mixture” as used herein typically refers to a population of heterogeneous cells obtained from a tissue or tumour biopsy. Where the biopsy is a tumour biopsy, the biopsy may be obtained from a primary tumour. Where the biopsy is from a tissue, the tissue may comprise a cancerous tissue. The cell mixture as disclosed herein typically comprises a cancer cell, a cancer stem cell, a stromal cell, a cancer-associated fibroblast, an endothelial cell, a pericyte and/or an epithelial cell, or a combination thereof.

As will be appreciated by the skilled addressee, the types and proportions of cells present in the cell mixture (and the tumour construct) are dependent upon the nature of the primary tumour (and the determined cellular profile of the tumour biopsy). As such, the types of cells (e.g. cancer cell, cancer stem cell, cancer-associated fibroblast etc.) may vary in accordance with the type of cancer microenvironment being mimicked.

Representative examples of cancer cells may include, but are not limited to, brain cancer cells, lung cancer cells, breast cancer cells, prostate cancer cells, colorectal cancer cells, ovarian cancer cells, pancreatic cancer cells, skin cancer cells, bone cancer cells, head and neck cancer cells, rare cancer cells, paediatric cancer cells, and the like. For example, the cancer cell may be selected from the group consisting of brain tumour cells, breast cancer cells and lung cancer cells.

Representative examples of cancer stem cells include, but are not limited to, brain cancer stem cells, lung cancer stem cells, breast cancer stem cells, prostate cancer stem cells, colorectal cancer stem cells, ovarian cancer stem cells, pancreatic cancer stem cells, skin cancer stem cells, bone cancer stem cells, head and neck cancer stem cells, rare cancer stem cells, paediatric cancer stem cells, and the like. For example, the cancer stem cell may be selected from the group consisting of brain tumour stem cells, breast cancer stem cells and lung cancer stem cells. In some examples, the cancer stem cell may be a glioblastoma cancer stem cell (GBM CSC). In other examples, the cancer stem cell may be a breast cancer stem cell (BCSC).

Representative examples of cancer associated fibroblast cells include, but are not limited to, brain cancer associated fibroblast cells, lung cancer associated fibroblast cells, breast cancer associated fibroblast cells, prostate cancer associated fibroblast cells, colorectal cancer associated fibroblast cells, ovarian cancer associated fibroblast cells, pancreatic cancer associated fibroblast cells, skin cancer associated fibroblast cells, bone cancer associated fibroblast cells, head and neck cancer associated fibroblast cells, rare cancer associated fibroblast cells, paediatric cancer associated fibroblast cells, and the like. For example, the cancer associated fibroblast cell may be selected from the group consisting of brain tumour associated fibroblast cells, breast cancer associated fibroblast cells and lung cancer associated fibroblast cells. In some examples, the cancer associated fibroblast cell may be a glioblastoma cancer associated fibroblast cell (GBM CAF). In some examples, the cancer associated fibroblast cell may be a breast cancer associated fibroblast cell (BC CAF).

By way of further example only, where the construct is to mimic the in vivo microenvironment of a brain tumour, the cell mixture and/or construct may comprise, consist essentially or consist of one or more of the following cell types: a brain tumour cell, a brain tumour stem cell, a brain tumour associated fibroblast cell, astrocyte and microglia, in addition to the immune cells as described herein.

By way of additional example, where the construct is to mimic the in vivo microenvironment of a breast cancer, the cell mixture and/or construct may comprise, consist essentially or consist of one or more of the following cell types: a breast cancer cell, a breast cancer stem cell, a breast cancer associated fibroblast cell and an adipocyte cell (such as a human adipocyte cell), in addition to the immune cells as described herein.

The cell mixture may comprise (or be formed from) a population of cells as described above, wherein each cell type may be included in a proportion that facilitates and/or promotes the growth and/or development of a construct that mimics the in vivo cancer microenvironment. Preferably, each cell type may be included in a proportion that mimics the in vivo cancer microenvironment, which may be inferred by the proportion of each cell type identified in the tumour biopsy, for example.

In some examples, the cell mixture may comprise the same (or substantially the same) proportion or percentage of each cell type identified in the tumour biopsy. In other words, the cell composition (e.g. cell percentage or proportion of each cell type relative to the total number of cells in a sample) of the cell mixture may be proportionate to the cell composition of the tumour biopsy.

Without wishing to be bound by theory, as the present disclosure provides a personalised approach to modelling a tumour microenvironment, the number of each cell type of the cell mixture may depend on the biopsy. As discussed herein, the skilled person will recognise that the cellular profile of the tumour biopsy (e.g. number and/or percentage of each cell type present within the tumour biopsy) may be determined by existing methods known in the art, such as FACS for example. In some instances, it may be assumed that approximately 50% (or more) of the cells of the prepared cell mixture will die due to the stress placed on the cells during the depositing or printing step. Therefore, the skilled person may supplement the cell mixture with an additional at least about (or about) 50%, 60%, 70%, 80%, 85%, 90%, 95%, 100%, 125% or 150% of the desired cells to compensate for any cells that may be lost during the process of making the tumour construct.

In some examples, cancer cells of the cell mixture or tumour construct may comprise between about 20% and 90%, between about 30% and 80%, between about 40% and 70%, between about 45% and 65%, or between about 50% and 60% of the cell population. In some examples, cancer cells may comprise between about 60% to 80%, or between about 65% and 75% of the cell population (e.g. about 70% of the cell population).

In some examples, cancer stem cells of the cell mixture or tumour construct may comprise between about 0.5% and 5%, between about 1% and 2.5%, or about 2% of the cell population. In some examples, cancer associated fibroblast cells of the cell mixture or tumour construct may comprise between about 2.5% and 15%, or between about 5% and 10% of the cell population. In some examples, cancer associated fibroblast cells may comprise between about 2.5% and 7.5% of the cell population.

In addition, or alternatively, the composition of the immune cells derived (or obtained) from or provided by the subject used to (optionally) supplement the cell mixture or the tumour construct, may be proportionate to the immune cell composition of the tumour biopsy. Without wishing to be bound by theory, the optional supplementation of the cell mixture and/or tumour construct with the immune cells derived from the patient substantially recapitulates or replicates the immune cell profile of the tumour biopsy.

As will be understood from the above, the immune cells of the present disclosure are typically obtained from the subject and/or patient. In some examples, the immune cells comprise white blood cells. In some examples, the immune cells comprise peripheral mononuclear blood cells (PBMCs) or cells derived therefrom. In some examples, the PBMCs may be further differentiated into NK cells, T cell, macrophages, B cells, monocytes and/or dendritic cells, or a combination thereof. In some examples, the PBMCs may be further differentiated into NK cells, T cells and/or macrophages, or a combination thereof. The differentiation may be conducted by following the differentiation protocols of the present disclosure (see Example Methods), for example. For the avoidance of doubt, the term “PBMCs or cells derived therefrom” may be used to encompass any PBMCs or cells that are (further) differentiated from PBMCs.

In some examples, the method may further comprise obtaining a sample of a bodily fluid from a subject or patient, wherein said bodily fluid may comprise immune cells. In some examples, the method may comprise obtaining a blood sample from a subject or patient (e.g. the same patient or subject from whom the tumour biopsy was obtained). In other words, the method may comprise providing or obtaining a patient-matched blood sample. In yet further examples, the method may comprise isolating the immune cells from the sample obtained from (or provided by) the patient or subject and using these isolated cells to supplement the cell mixture (as is described in more detail below).

In some examples, a tumour construct may be prepared from a cell mixture as disclosed herein and the immune cell composition of the tumour construct may be assessed at various time points (e.g. day 0 (the day the tumour construct is made, deposited or printed), day 7 and day 14) in order to determine the extent of depletion of each cell type over time. Subsequently, the tumour construct may be supplemented using the immune cells isolated from the same subject from whom the tumour biopsy was obtained or provided by.

As such, in some examples, the PBMCs derived from or provided by the subject or patient may be used to optionally supplement the cell mixture and/or the tumour construct.

By way of example, sterile immune cells may be isolated from matched peripheral blood mononuclear cells (PBMCs) via fluorescence activated cell sorting (FACS) on the day of depositing or (bio)printing the tumour construct. The isolated PBMCs may be used to supplement the cell mixture or the tumour construct comprising the cells obtained from the biopsy of the primary tumour. For example, specific immune cell populations may be isolated from PBMCs based on the expression profile using a cell sorter by using cell-specific markers known in the art, such as those provided in the table below.

| Cell Type: | Marker Expression: |
|---|---|
| T Cells | CD3⁺ |
| T Cells (Helper) | CD3⁺/CD4⁺ |
| T Cells (Cytotoxic) | CD3⁺/CD8⁺ |
| T Cells (Regulatory) | CD3⁺/CD25⁺ |
| T Cells (Activated) | CD3⁺/HLA-DR⁺ |
| T Cell (Inhibitory) | CD3⁺/CD274(PDL-1)⁺ |
| Leucocytes | CD45⁺ |
| NK Cells | CD16⁺ |
| B Cells | CD19⁺/CD22⁺ |
| Macrophages | CD14⁺ |

In some examples, PMBC-derived monocytes may be further differentiated into NK cells, B cells, T cell, macrophages and/or dendritic cells. The differentiated immune cells may be used to supplement the cell mixture prior to making the tumour construct or the tumour construct may be supplemented with the differentiated immune cells.

Without wishing to be bound by theory, viability, the immune cell function and/or the composition of the immune cells of the tumour construct is/are maintained for at least 7 days, at least 14 days or at least 21 days. In some examples, the viability, the immune cell function and/or the composition of the PBMCs or cells derived therefrom maintained for at least 7 days, at least 14 days or at least 21 days.

A cell mixture of the present disclosure (which cell mixture may be prepared by any of the expansion steps described herein (which expansion step uses any of the media (e.g. expansion media) described herein)) may further comprise a bioink and/or one or more growth factors.

In the broadest sense, the term “bioink” encompasses any suitable material that may be used for making a tumour construct, depositing a tumour construct, and/or (bio)printing a tumour construct e.g. in a 2D and/or 3D configuration.

The term “bioink” as used herein may refer to any natural or synthetic polymer selected for its biocompatible components and favourable rheological properties.

By way of example, the bioink may be biocompatible, may facilitate mixing, may be extrudable and/or may support cell growth and/or development. Such bio-inks may mimic the extracellular matrix environment and provide support for cell functions such as adhesion, proliferation, and differentiation after printing. It should be appreciated that bio-ink formulations may vary depending on the cell type.

As such, bioink may comprise one or more extracellular matrix components. The extracellular matrix component may be selected from any suitable material that can be extruded in the deposition/printing step, that is biocompatible with the cells present in the construct and/or that can support the growth and/or development of the construct.

Suitable matrix materials may include but are not limited to hydrogels, naturally occurring polymers and synthetic polymers. The matrix may be or comprise a hydrogel, for example, a collagen, gelatin, fibrin, polyethylene and/or polysaccharide (e.g. hyaluronic acid, agarose, chitosan, or alginate)-based hydrogel.

Suitable cancer cell compatible materials (based on hydrogels) may include those outlined in the table below.

| BioInk | Material |
|---|---|
| Collagen PREMIUM | Collagen type I |
| Chitoink | Chitosan |
| ColMa | methacrylated collagen |
| Cell-link Laminink | Laminins-basal lining of cell |
| | membrane |
| GelXA laminink | Same as above but more |
| | stable at room temp |
| GelXG | Gelatin-based with Xanthan |
| | gum |
| Alginate | Alginate |
| GelMA High | gelatin methacrylate |
| GelMA-Alginate | gelatin methacrylate |
| GelMA HA | gelatin methacrylate |
| Gel XA | GelMA base, xanthan gum |
| | and alginate |
| Coll 1 | Collagen type I |
| GelMA A | methacrylated collagen and |
| | alginate |
| GelMA C | |
| Cellink A | Sodium alginate |
| Celllink RGD | Polysaccharide hydrogel and |
| | RGD |
| Cellink A RGD | Sodium alginate and RGD |
| Cellink bioink | Polysaccharide hydrogel |

In certain examples, the matrix may be or comprise one or more of: an alginate-based material (e.g. sodium alginate), cellulose (e.g. nanofibrillar cellulose); and extracellular proteins (e.g. laminin). A representative example of a suitable extracellular matrix is Cellink Laminink 411 (obtained from Cellink LifeSciences). The composition of Laminink 411 is set out below.

| Chemical name | CAS# | EC No. | EC Class |
|---|---|---|---|
| Nanofibrillar Cellulose | 9004-34-6 | None | Not classified as hazardous |
| Sodium Alginate | 9005-38-3 | None | Not classified as hazardous |
| D-mannitol | 69-65-8 | 200-711-8 | Not classified as hazardous |
| Laminin 411 | N/A | None | Not classified as hazardous |
| HEPES buffer solution | 7365-45-9 | 230-907-9 | Not classified as hazardous |

A further example of a suitable extracellular matrix may be GrowInk™ (obtained from UPM Biomedicals), a hydrogel based matrix comprising nanofibrillar cellulose and optionally alginate.

In some examples, the matrix may be a decellularized extracellular matrix material. By way of example, the decellularized extracellular matrix material may be derived from a subject or a patient (in some examples, the same subject or patient who provided the tumour biopsy). The term “using said supplemented cell mixture to prepare a tumour construct”, “using said cell mixture to prepare a tumour construct” or “using the expanded cells to prepare a tissue or tumour construct” may refer to depositing said mixture or expanded cells. The step of depositing may be carried out by any suitable technique that facilitates the placement of the components of the desired construct (e.g. tumour construct or tissue construct) at a precise location. Representative examples include, but are not limited to, printing (e.g. (bio)printing), spreading, pipetting, spraying, or coating on to the surface. In some examples, the step of depositing comprises printing (e.g. (bio)printing) the cells and the extracellular matrix on to the surface. In some examples, the cell mixture may comprise spheroids which may be maintained in suspension and thus, does not necessarily require the cell mixture to be deposited or printed onto a surface.

Cells, for example dissociated cells, prepared according to any of the methods described herein may be combined with any of the bioinks described herein. The expanded cell, bioink mix may then be used for bioprinting the required (tissue or tumour) constructs. As stated, the cells (e.g. dissociated cells) may be expanded so that they reach a number which is suitable for bioprinting (e.g. 11-12 million). Those cells may then then be combined with the bioink according to a predetermined ratio. For example the expanded (dissociated) cells may be combined with bioink (to for a cell/bioink mix) at a ratio of 50,000-500,000 cells per 1-10 μl of bioink. For example 75,000, 100,000, 125,000, 150,000, 175,000, 200,000, 225,000, 250,000, 275,000, 300,000, 325,000, 350,000, 375,000, 400,000, 425,000, 450,000 or 475,000 cells may be combined with 2, 3, 4, 5, 6, 7, 8 or 9 μl of bioink. In one teaching, the expanded or dissociated cells, may be combined with the bioink (to form a cell/bioink mix) at a ratio of 100,000 cells per 3 ul of bioink. An additional volume of bioink may be added to allow for waste during mixing processes. The additional volume may be added according to the following calculation: approximately 100 μl bioink per volume (e.g 3 μl) cell/bioink mix.

As used herein, printing may refer to the three dimensional printing of biological material(s). Printing (or (bio)printing) may comprise any suitable technique to deposit the cell mixture, and optionally immune cells, in or at the desired location on a surface. In addition, or optionally, a bioink, one or more growth factors and/or one or more extracellular matrix components may be deposited or printed with the cell mixture.

The printing may comprise an extrusion based printing technique. By way of example only, the described methods may employ pneumatic printing (which involves extruding a material using air pressure) or may employ a syringe printhead. Printing using a syringe printhead involves mechanically applied pressure to a syringe plunger. The use of a syringe printhead may be helpful to increase consistency of droplet size when working at much smaller volumes, but it can be more challenging to control the shear stress experienced by the cells.

The printing step may comprise printing a plurality of constructs at a series of defined and/or discrete locations on a surface e.g. in a predetermined pattern to provide an array or microarray of constructs. By way of example only, the method may comprise depositing or printing the bioink in a plurality of wells on a multi-well plate, e.g. a 96-well plate or a 384-well plate.

In any of the above described methods and constructs, the cell mixture, and optionally the immune cells, are deposited (e.g. printed) onto the surface to provide the construct. The surface may define a series of locations (e.g. wells) upon which the components may be deposited. Suitable surfaces may be any surface that is compatible (e.g. biocompatible) with the components, is chemically inert and/or that facilitates the culture and/or growth of the construct after deposition. The surface may be or comprise glass, plastic and/or a polymeric material. In some examples, the surface may be or comprise PDMS (polydimethylsiloxane), polycarbonate, or the like.

In particular, the methods disclosed herein may comprise printing droplets of a bioink on to the surface. Within the context of the present disclosure, the bioink may be considered to be made up of the cells obtained from a tumour biopsy obtained from or provided by a subject and optionally immune cells derived from the same subject, which may be pre-mixed with and/or suspended in the extracellular matrix.

The method may comprise culturing cells obtained from a tumour biopsy to provide spheroids prior to the deposition or printing step. In particular, the method may comprise co-culturing cells obtained from a tumour biopsy to provide spheroids prior to the deposition or printing step. Thus, the described methods may involve depositing or printing spheroids on to the surface.

The use of multicellular spheroids (composed of the three or more cell types discussed here) in the deposition or printing step may assist in making the resultant constructs more complex, dense and closer to the in vivo cancer microenvironment.

Alternatively, the method may comprise depositing or printing cells on to the surface. Such methods may be referred to as a “single cell” printing methods herein. Single cell printing methods may offer a particularly consistent method of providing the constructs and/or may provide constructs of greater consistency.

Where the method comprises depositing or printing cells on to the surface, spheroids may form in situ in the deposits. For example, the cells within the deposits may be cultured for a period of time and/or in a suitable culture medium to facilitate the formation of spheroids (e.g. three-dimensional spheroids).

In other examples, the method may comprise depositing or printing a mixture of spheroids and single cells on to the surface.

The use of spheroids within the deposited or printed constructs (whether these are formed before or after the deposition/printing step) can assist in providing a more accurate representation of the in vivo cancer microenvironment as they facilitate cell-cell interactions and/or cell-ECM interactions in a three-dimensional structure.

As used herein, a spheroid (or three-dimensional spheroid) may refer to a three-dimensional cellular aggregate. In some cases, these aggregates may be generally spherical in nature. In a typical two-dimensional monolayer cell culture, cells tend to interact with the substrate upon which they are cultured. In contrast, in a spheroid, cells may be able to grow and/or interact with their surroundings in all three directions and/or a spheroid may enhance cell to cell interactions.

Any suitable technique may be employed for the production of the spheroids as described herein. Representative examples include, but are not limited to, the use of low cell adhesion plates (e.g. where the spheroids form in the rounded bottoms of multi-well plates) and the hanging drop method (e.g. where spheroids form in drops that hang from the surface of a cell plate).

By way of example only, the spheroids may be produced by co-culturing the cells of the cell mixture in a low adhesion plate for a pre-determined period of time (e.g. from 0 days to 30 days, such as from 1 day to 21 days, or from 2 days to 14 days) prior to deposition or printing. The cells may be cultured with a medium that promotes the formation of a spheroid (or 3D spheroid). By way of example only, the medium may comprise 3D Tumorsphere medium (obtained from PromoCell, GmbH).

In some examples, the described methods may further comprise a step of magnetic (bio)printing.

As used herein, magnetic (bio)printing may refer to a printing technique which involves the use of magnetic nanoparticles to print cells into a three-dimensional structure or pattern. In such methods, cells may be tagged with magnetic nanoparticles. External magnetic forces may then be applied to print the cells into a desired three-dimensional structure. Representative examples of magnetic nanoparticles include iron oxides (such as particles consisting essentially of gold, iron oxide and poly-L-lysine e.g. NanoShuttle-PL obtained from Greiner Bio-One).

Magnetic (bio)printing may be used to print the cells into spheroids prior to a printing or deposition step as described herein. Additionally, or alternatively, magnetic (bio)printing may be used to print cells into three-dimensional spheroids after deposition or printing of the bioink on to the surface.

The combination of extrusion based printing and magnetic based (bio)printing can assist in providing constructs that mimic an in vivo cancer microenvironment more rapidly and/or can provide a construct that more closely resembles a clinical sample.

In any of the described methods, the cell mixture and/or the spheroids (if used) comprising cells from a tumour biopsy may be mixed with the extracellular matrix prior to the deposition or printing step. In other words, prior to the printing or deposition step, the cells and/or spheroids may be mixed with the extracellular matrix to provide the bioink formulation.

Following the deposition or printing step, the method may further comprise cross-linking the constructs. The cross-linking step may be useful to provide a degree of rigidity and/or stiffness to the construct. The degree of rigidity and/or stiffness may depend on the nature of the cancer microenvironment that is being mimicked by the construct.

The cross-linking step may be carried out using methods known in the art. In some examples, the cross-linking may be carried out using a cross-linking agent. Suitable cross-linking agents may include metal salts (e.g. calcium chloride). In some examples, the cross-linking step may be light (UV or visible) dependent and/or temperature dependent.

Following the deposition step, the method may then comprise culturing the construct for a period of time such that the construct mimics an in vivo cancer microenvironment. In particular, the construct may be cultured in a medium and/or for a period of time to allow the cells to grow and/or develop so that the construct more closely reflects an in vivo cancer microenvironment. At this stage, the construct may be considered ready for use as a cancer model e.g. in drug testing or development.

In some examples, the construct may be cultured for a period of time anywhere between 1 day and 30 days, in other examples, the construct may be cultured for a period of time anywhere between 2 days and 21 days, or between 3 days and 18 days. In some examples, the construct may be cultured for a period of time between 14 days and 21 days. The construct may be cultured in any suitable medium (e.g. a culture medium) that promotes growth and/or development of the three-dimensional construct.

The culture medium may comprise growth factors, developmental factors, supplements, buffers and/or other components to promote the growth and/or development of the construct such that it mimics an in vivo cancer microenvironment. By way of example only, the medium may comprise 3D Tumorsphere medium (obtained from PromoCell, GmbH). Alternatively, or additionally, the medium may comprise fetal bovine serum solution (e.g FBS replacement solution (Hyclone FBS)), ascorbic acid and/or epidermal growth factor (EGF) growth factor. By way of representative example only, a culture medium may comprise 30 μl Hyclone FBS, 25 ng/ml epidermal growth factor (EGF), and 50 μg/ml ascorbic acid (AA).

The cells and/or spheroids may be mixed with the culture medium prior to the deposition/printing step. In particular, the cells and/or spheroids may be suspended in the culture medium to provide a suspension of cells and/or spheroids prior to the deposition or printing step. In those cases where an extracellular matrix component is present, the method may comprising mixing the suspension of cells and/or spheroids with the extracellular matrix to provide a bioink formulation that is used in the printing step.

Within the bioink formulation, the relative proportions of the suspension of cells and/or spheroids and the extracellular matrix may be carefully controlled to facilitate the deposition and/or printing step. The relative proportions of the suspension of cells and/or spheroids and the extracellular matrix may be selected dependent upon on the particular cancer environment that is being mimicked by the construct.

In some examples, between about 10% and 90% of the total volume of the bioink formulation is comprised of the suspension of cells and/or spheroids. In some examples, between about 40% and 80% of the total volume of the bioink formulation is comprised of the suspension of cells and/or spheroids. In certain cases, between about 45% and 75% of the total volume of the bioink formulation is comprised of the suspension of cells and/or spheroids. In some examples, between about 90% and 10% of the total volume of the bioink formulation is comprised of the extracellular matrix. In some examples, between about 60% and 20% of the total volume of the bioink formulation is comprised of the extracellular matrix. In certain cases, between about 55% and 25% of the total volume of the bioink formulation is comprised of the extracellular matrix.

In some examples, between about 40% and 80% of the total volume of the bioink formulation is comprised of the suspension of cells and/or spheroids and between about 60% and 20% of the total volume of the bioink formulation is comprised of the extracellular matrix. The use of such ratios can enhance and/or promote cell proliferation within the constructs and/or can provide stable constructs.

In one case, the bioink formulation may comprise approximately 70% by volume of the suspension of cells and/or spheroids and approximately 30% by volume of the extracellular matrix. In another example, the bioink formulation may comprise approximately 50% by volume of the suspension of cells and/or spheroids and approximately 50% by volume of the extracellular matrix.

In view of the above, according to a yet further aspect of the disclosure, there is provided a bioink formulation for use in any of the methods described herein. For the avoidance of doubt, the bioink formulation may be used for (i) (bio)printing a supplemented cell mixture generated by a method of this invention; or (ii) for (bio)printing cells that have been expanded using any of the media described herein.

The bioink formulation may comprise:
(a) one or more polymers; and/or
(b) one or more extracellular matrix components; and/or
(c) a cell mixture obtained from a tumour biopsy obtained from or provided by a subject; and optionally further comprising:
(d) immune cells obtained from or provided by said subject; and/or
(e) endothelial cells; and/or
(f) one or more growth factors.

It will be appreciated that the bioink formulation may be in a form that is extrudable and/or that is suitable for deposition or printing.

Alternatively, a bioink formulation of the present disclosure may comprise:
(a) laminin, alginate, collagen, nanofibrillated cellulose and/or fibrinogen, or a combination thereof;
(b) a cell mixture prepared according to any of the methods described herein or from a tumour biopsy obtained from or provided by a subject; and optionally,
(c) immune cells obtained from said subject.

The cell mixture and the immune cells of any of the bioink formulations may comprise a cell composition as disclosed herein. The cell mixture may comprise cells which have been subject to an expansion technique comprising any of the media compositions (e.g. expansion media) described herein.

Further, the present disclosure provides the described constructs for use in drug development, drug screening and/or clinical evaluation of a drug product or a treatment.

In particular, an active agent or a treatment may be applied to and/or contacted with the described constructs which are designed to mimic an in vivo microenvironment (e.g. an in vivo cancer microenvironment). Characteristics of the in vivo cancer microenvironment may be monitored to assess the effect and/or activity of a compound. For example, cell proliferation and/or cell viability may be monitored in the in vivo cancer microenvironment to determine the efficacy of a compound.

Accordingly, in one aspect of the present disclosure, there is provided a construct for mimicking an in vivo tumour environment, the construct comprising:
(a) patient-derived cells from a tumour biopsy obtained from or provided by a subject, wherein said patient-derived cells comprise a cancer cell, a cancer stem cell, a stromal cell, a cancer-associated fibroblast, an endothelial cell, a pericyte and/or an epithelial cell, or a combination thereof; and
(b) immune cells derived from or provided by said subject.

The constructs of this disclosure may be used to permit the testing of agents, for example drugs, on cells. The constructs allow a user to monitor and determine the response of a cell to a treatment, such as a test agent or drug, or a combination of treatments. An advantage of the constructs disclosed herein is that by maintaining cells in (micro) environments/niches which replicate aspects of the in vivo (micro) environments/niches, the cells will respond to the treatment in a way that better represents how the cells might respond to those test agents/drugs in vivo.

According to another aspect of the disclosure, there is provided a method of testing cell response to a treatment, said method comprising:
(a) providing a tumour construct made according to any of the methods or constructs disclosed herein;
(b) contacting the tumour construct with the treatment;
(c) optionally, maintaining the tumour construct with the treatment; and
(d) determining the response of one or more cells to the treatment.

Optionally, the above method may further comprise characterising one or more cells of the tumour construct prior to and/or subsequent to contacting the tumour construct with the treatment.

In particular, a treatment may be applied to and/or contacted with the described constructs which are designed to mimic an in vivo cancer microenvironment. Characteristics of the in vivo cancer microenvironment may be monitored to assess the effect and/or activity of a treatment. For example, cell proliferation and/or cell viability may be monitored to determine the efficacy of a treatment. Exemplary assays that may be used to assess cell response to a treatment or an active agent may include a cell viability assay, cell cytotoxicity assay, apoptosis assay, hypoxia assay and/or a cytokine array, for example.

The treatment may be selected from (but are not limited to): a compound, a drug, an antibody, radiation therapy, ultrasound therapy, radiofrequency therapy, laser therapy, UV therapy, photodynamic therapy, electrochemotherapy, immunotherapy, stem cell therapy, heat therapy, cryotherapy and/or therapeutic oligonucleotides. The treatment may be a combination of treatments, e.g. two or more treatments optionally selected from those described above. Where a combination of treatments is contacted with the tumour construct, these may be contacted (e.g. applied) sequentially or concurrently to the construct.

According to a further aspect of the present disclosure, there is provided a kit comprising a cell mixture obtained from a tumour biopsy and a population of immune cells obtained from a blood sample. Preferably, the cells of the cell mixture obtained from a tumour biopsy and the immune cells are obtained from the same patient or subject. The cell mixture provided as part of the kit may comprise single cells and/or spheroids. These cell types may be supplied and/or stored separately within the kit. Alternatively, the various components may be provided in the kit as a bioink formulation as described herein.

The kit may further comprise a culture medium and/or instructions for use. In addition, the kit may optionally comprise one or more extracellular matrix components, one or more growth factors, and/or one or more endothelial cells. Optionally, the kit may further comprise a substrate defining a deposition or print surface.

In some examples, the kit may be cryopreserved for storage and/or delivery. The cryopreserved components of the kit may comprise a cell mixture obtained from a tumour biopsy and/or a population of immune cells obtained from a blood sample. The cell mixture obtained from a tumour biopsy and said immune cells may be cryopreserved or maintained together or separately for storage and/or delivery. Cryopreservation may be conducted using the protocol provided in the Example Methods section of the present disclosure or any other suitable cryopreservation methods known in the art.

As detailed herein, a tissue or tumour sample/biopsy is dissociated using enzymatic and mechanical methods. The composition (e.g. cell composition) of the tissue/tumour sample/biopsy may then be assessed by immunohistochemistry, immunofluorescence and flow cytometry. The dissociated cells may then be maintained and expanded in culture with the aim of obtaining a suitable number of cells for (bio)printing.

As it would be appreciated by the skilled person in the art, the maintenance and expansion of a heterogeneous population of cells obtained from a tissue (e.g. a tumour biopsy) is a crucial, but challenging, part of providing an artificial (e.g. bio-printed) construct which mimics the in vivo state (e.g. the original tumour). In this context the term ‘mimic’ may mean that a bio-printed construct comprises substantially the same cells and/or the same cellular organisation as the tissue/tumour from which the sample/biopsy (and ultimately the (bio)printed construct) is derived.

Unlike immortalised cell lines, primary cells typically have a limited lifespan and can only be maintained in vitro for a limited amount of time. This is especially true of the immune cell component of a tissue or tumour. Accordingly, when developing the tissue/tumour construct manufacturing methods of this disclosure, there was a need to develop a method or culture condition which supports the culture, maintenance, expansion and/or propagation of a heterogeneous population of primary cells in vitro. The aim is to expand cells dissociated from a tissue or tumour sample/biopsy into a population which comprises a number of cells sufficient to permit a the (bio)printing of an artificial construct (e.g. tumour) which mimics the original tissue/biopsy (e.g. tumour/tumour biopsy).

In prior experimentation by the present inventors, the use of a standard (prior art) tissue culture medium, for example a medium comprising DMEM/F12, Fetal Hyclone 1 (10%) and Penicillin/Streptomycin 1% failed to yield an expanded cell population that was suitable for (bio)printing. The use of a standard medium necessitated extended cell culture which increases the risk of cell loss through cell death. By way of example, the use of a standard medium frequently required a cell culture period of 6-8 weeks (tissue dependent) to reach a sufficient number of cells required for (bio)printing (e.g. 11-12 million cells) as well as loss of cellular components, such as tumour-infiltrating immune cells.

To resolve these issues, the present inventors have developed a culture media composition which represents an improvement over the prior art as it shortens the time required to achieve an expanded cell population for (bio)printing, avoids excessive cell loss and preserves the overall composition (e.g. cell proportions and cell types) of the original sample (e.g. tissue/tumour sample or biopsy).

Accordingly, in a further aspect there is provided a cell culture medium (which may be used in the preparing step of any of the methods described herein) for the maintenance and/or expansion of cells obtained or dissociated from a sample or biopsy (e.g. a tissue sample or biopsy), said medium comprising:
(a) one or more growth factors; and/or
(b) one or more glucocorticoids;
and optionally,
(c) a serum supplement; and/or
(d) a vitamin supplement; and/or
(e) one or more antibiotics.

In one teaching, the medium of present disclosure may comprise:
(a) one or more growth factors; and/or
(b) one or more glucocorticoids; and/or
(c) a serum supplement; and/or
(d) a vitamin supplement; and/or
(e) one or more antibiotics.

In one teaching, the medium of present disclosure may comprise:
(a) one or more growth factors, wherein said one or more growth factors include insulin, EGF and FGF; and/or
(b) one or more glucocorticoids, wherein said one or more glucocorticoid(s) include hydrocortisone; and/or
(c) a serum supplement; and/or
(d) a vitamin supplement; and/or
(e) one or more antibiotics.

In one teaching, the medium of present disclosure may comprise:
(a) one or more growth factors, wherein the one or more growth factors include insulin, EGF and FGF; and/or
(b) one or more glucocorticoids, wherein said one or more glucocorticoids include hydrocortisone.

In some examples, the medium of the present disclosure may comprise:
(a) Insulin; and/or
(b) FGF; and/or
(c) EGF; and/or
(d) Hydrocortisone; and/or
(e) Fetal bovine serum or Fetal Hyclone 1; and/or
(f) MEM Vitamin Solution; and/or
(g) Penicillin/Streptomycin.

In some examples, a medium of the present disclosure may comprise at least two components selected from the group consisting of:
(a) Insulin;
(b) FGF;
(c) EGF;
(d) Hydrocortisone;
(e) Fetal bovine serum or Fetal Hyclone 1;
(f) MEM Vitamin Solution; and
(g) Penicillin/Streptomycin.

In some examples, a medium of the present disclosure may comprise at least three components selected from the group consisting of:
(a) Insulin;
(b) FGF;
(c) EGF;
(d) Hydrocortisone;
(e) Fetal bovine serum or Fetal Hyclone 1;
(f) MEM Vitamin Solution; and
(g) Penicillin/Streptomycin.

In some examples, a medium of the present disclosure may comprise at least four components selected from the group consisting of:
(a) Insulin;
(b) FGF;
(c) EGF;
(d) Hydrocortisone;
(e) Fetal bovine serum or Fetal Hyclone 1;
(f) MEM Vitamin Solution; and
(g) Penicillin/Streptomycin.

In some examples, a medium of the present disclosure may comprise at least five components selected from the group consisting of:
(a) Insulin;
(b) FGF;
(c) EGF;
(d) Hydrocortisone;
(e) Fetal bovine serum or Fetal Hyclone 1;
(f) MEM Vitamin Solution; and
(g) Penicillin/Streptomycin.

In some examples, a medium of the present disclosure may comprise:
(a) Insulin;
(b) FGF;
(c) EGF;
(d) Hydrocortisone;
(e) Fetal bovine serum or Fetal Hyclone 1;
(f) MEM Vitamin Solution; and
(g) Penicillin/Streptomycin.

In another example, a medium of the present disclosure may comprise:
(a) Insulin;
(b) FGF;
(c) EGF; and
(d) Hydrocortisone.

In any of the examples provided herein, FGF may be bFGF. Similarly, in any of the examples provided herein, EGF may be bEGF.

Additionally, or alternatively, Fetal Hyclone 1, rather than Fetal bovine serum, may be used when processing samples obtained from a patient.

It should be understood a medium of the present disclosure (which may be referred to as an “expansion medium”) may comprise any of the supplementary components described herein including any of those listed as components (a) to (g) above, individually or in combination.

Without wishing to be bound by theory, and by way of example only, the exemplary supplementary components of a medium of the present disclosure may have the functions detailed below.

Insulin may act as a growth factor and the main growth-promoting effect of insulin is through its low-affinity interaction with the insulin-like growth factor I receptor (IGF-IR). IGF-IR may be involved in regulation of cell growth and metabolism.

Fibroblast growth factors signal through FGF receptors and may regulate a wide range of biological functions such as cell proliferation, cell survival, cell differentiation and migration. Epidermal growth factor is a mitogen, which may help to promote cell proliferation by inducing cell division. EGF may also delay the onset of cell senescence.

Hydrocortisone aids cell growth for specific cell types, such as endothelial and epithelial cells.

Fetal bovine serum (FBS) may be used to supplement media containing multiple growth promoting factors. These include but are not limited to: amino acids, proteins, vitamins carbohydrates, lipids, hormones, growth factors, minerals, and trace elements. For supplemented medium for culturing patient samples, Fetal Hyclone 1 may be used. FBS may be preferred when culturing cell lines.

The medium of the present disclosure may comprise a MEM vitamin solution. By way of example only, MEM Vitamin Solution 100× may be used, which contains 100× the concentration of the standard vitamins found in regular Modified Eagles medium. Cells cultured in vitro may not be able to synthesise vitamins in suitable amounts for good growth and proliferation, and therefore vitamins may be provided in the cell culture medium.

The combination of penicillin/streptomycin reduces the risk of microbial contamination spoiling a cell culture. It acts by binding to the 30S subunit of the bacterial ribosome, leading to inhibition of protein synthesis and death in susceptible bacteria. It is useful in primary cell culture as donor tissue can be a potential source of contamination.

A medium of the present disclosure may comprise:
(a) Insulin: 5.0-15.0 μg/mL;
(b) FGF: 0.001-0.010 μg/mL;
(c) EGF: 0.001-0.010 μg/mL;
(d) Hydrocortisone: 0.1-0.3 μg/mL;
(e) Penicillin/Streptomycin: 80.0-120.0 μg/mL; and
(f) optionally, a serum supplement and/or a vitamin supplement.

A medium of the present disclosure may comprise:
(a) one or more growth factors, wherein the one or more growth factors include:
   i. Insulin: 5.0-15.0 μg/mL;
   ii. FGF: 0.001-0.010 μg/mL;
   iii. EGF: 0.001-0.010 μg/mL;
and/or
(b) one or more glucocorticoids, wherein said one or more glucocorticoids include: Hydrocortisone: 0.1-0.3 μg/mL; and
(c) optionally, a serum supplement and/or a vitamin supplement.

A medium of the present disclosure may comprise:
(a) Insulin: 5.0-15.0 μg/mL;
(b) FGF: 0.001-0.010 μg/mL;
(c) EGF: 0.001-0.010 μg/mL;
(d) Hydrocortisone: 0.1-0.3 μg/mL;
(e) optionally, Penicillin/Streptomycin: 80.0-120.0 μg/mL; and
(f) further optionally, a serum supplement and/or a vitamin supplement.

A medium of the present disclosure may comprise:
(a) Insulin: 5.0-15.0 μg/mL;
(b) FGF: 0.001-0.010 μg/mL;
(c) EGF: 0.001-0.010 μg/mL;
(d) Hydrocortisone: 0.1-0.3 μg/mL;
(e) Penicillin/Streptomycin: 80.0-120.0 μg/mL; and
(f) optionally, a serum supplement and/or a vitamin supplement.

A medium of the present disclosure may comprise one or a combination of supplementary components selected from the group consisting essentially of:
(a) Insulin: 10.0 μg/mL;
(b) FGF: 0.004 μg/mL;
(c) EGF: 0.003 μg/mL;
(d) Hydrocortisone: 0.2 μg/mL; and
(e) optionally, a serum supplement, a vitamin supplement and/or one or more antibiotics.

A medium of the present disclosure may comprise:
(a) Insulin: 10.0 μg/mL;
(b) FGF: 0.004 μg/mL;
(c) EGF: 0.003 μg/mL;
(d) Hydrocortisone: 0.2 μg/mL; and
(e) optionally, a serum supplement, a vitamin supplement and/or one or more antibiotics.

As stated, the serum supplement may be Fetal bovine serum or Fetal Hyclone 1.

An exemplary vitamin supplement may be MEM Vitamin solution.

An exemplary antibiotic combination may be penicillin/streptomycin.

A medium of the present disclosure may comprise:
(f) Insulin: 10.0 μg/mL;
(g) FGF: 0.004 μg/mL;
(h) EGF: 0.003 μg/mL;
(i) Hydrocortisone: 0.2 μg/mL;
(j) Fetal bovine serum or Fetal Hyclone 1;
(k) MEM Vitamin Solution; and
(l) Penicillin/Streptomycin.

A medium of the present disclosure may comprise one or a combination of components selected from the group consisting of:
(a) Insulin: 10.0 μg/mL;
(b) FGF: 0.004 μg/mL;
(c) EGF: 0.003 μg/mL;
(d) Hydrocortisone: 0.2 μg/mL;
(e) Penicillin/Streptomycin: 100.0 μg/mL; and
(f) optionally, a serum supplement and/or a vitamin supplement.

A medium of the present disclosure may comprise:
(a) Insulin: 10.0 μg/mL;
(b) FGF: 0.004 μg/mL;
(c) EGF: 0.003 μg/mL;
(d) Hydrocortisone: 0.2 μg/mL;
(e) Penicillin/Streptomycin: 100.0 μg/mL; and
(f) optionally, a serum supplement and/or a vitamin supplement.

A medium of the present disclosure may comprise:
(a) Insulin: 10.0 μg/mL;
(b) FGF: 0.004 μg/mL;
(c) EGF: 0.003 μg/mL;
(d) Hydrocortisone: 0.2 μg/mL;
(e) Penicillin/Streptomycin: 100.0 μg/mL;
(f) Fetal bovine serum or Fetal Hyclone 1; and
(g) MEM Vitamin Solution.

A medium of the present disclosure may comprise:
(a) Insulin: 10.0 μg/mL;
(b) FGF: 0.004 μg/mL;
(c) EGF: 0.003 μg/mL; and
(d) Hydrocortisone: 0.2 μg/mL.

In some examples, the medium of the present disclosure may be serum free.

In some examples, the medium of the present disclosure may be free of antibiotics, such as penicillin/streptomycin.

While the media described herein use DMEM/F12 as the base medium, an alternative base medium may be used.

As stated, the present inventors have found that the various media described herein reduce the period of time dissociated cells (obtained from a sample—e.g. a biopsy (e.g. tumour biopsy)) need to be maintained in culture in order to obtain a sufficient number of cells for (bio)printing. Where the maintained and expanded cells are to be used to (bio)print a tumour construct, the number of cells should be sufficient for that purpose.

As stated, the terms “biopsy” and “sample” may be interchangeable. Within the context of the present disclosure, a sample or a biopsy may be obtained from a tumour from a subject, such as a cancer patient.

As used herein, “a sufficient number of cells” for (bio)printing, e.g. (bio)printing a tumour construct is in the region of 5 million to 20 million cells, for example at least 6 million, 7 million, 8 million, 9 million, 10 million, 11 million, 12 million, 13 million, 14 million, 15 million, 16 million, 17 million, 18 million or at least 19 million cells. This number of cells may be sufficient for (bio)printing into a 96 well plate. The total number of cells needed will therefore depend on the number of constructs to be (bio)printed and one of skill may work on the assumption that at least 5-20 million (for example 11-12 million) cells are needed to successfully print a full 96 well plate. Of course the exact number of cells needed may further depend on the dimensions of each well to be printed into. Using the exemplary values provided above, the skilled person would be able to determine an appropriate number of cells needed to successfully print into any given plate.

The total number of cells expanded using a medium of this disclosure may be harvested, optionally washed and re-suspended in a quantity of (bio)print ink. For example, the maintained and expanded cells may be harvested and prepared as a 100 μL cell suspension.

In one example, the number of cells required for the (bio)print may be calculated by:

N=number wells×number of cells per well+200 ul more (to account for loss during (bio)printing or during preparation of the bioink cell mixture).

Without wishing to be bound by theory, it is hypothesised that the reduction in the time needed to expand the required number of cells, ensures that the expansion process occurs without compromising the composition of the cellular components present in the initial sample or biopsy obtained from a subject.

As used herein, the terms “cell culture period” or “maintenance period” or “expansion period” refer to the amount of time required for the cells obtained from a biopsy or a sample to be expanded to the number of cells sufficient for (bio)printing. These periods may be in the order of 2, 3, 4, 5, 6, 7 or 8 weeks. In some examples, the period may be at least 2 weeks. In some examples, the cell culture period may be at least 3 weeks. In some examples, the cell culture period may be at least 4 weeks. In some examples, the cell culture period may be at least 5 weeks. In some examples, the cell culture period may be at least 6 weeks. In some examples, the cell culture period may be at least 7 weeks. In some examples, the cell culture period may be at least 8 weeks. In some examples, the cell culture period may be 4 to 6 weeks.

As stated, the medium of the present disclosure promotes maintenance and/or expansion of the cells present in the initial sample or biopsy.

As used herein, the term “maintenance and/or expansion of dissociated cells” is typically characterised by retaining the cell composition and/or the proportion of each cell type present in the initial sample or biopsy obtained from a subject.

In one teaching, a medium of the present disclosure promotes maintenance and/or expansion of dissociated cells, wherein at least the cell composition of the initial sample or biopsy is maintained throughout the maintenance and/or expansion period in order to yield a cell population which is (a) suitable for (bio)printing and (b) representative of the cellular composition of the original sample and/or biopsy,

Cells types which are maintained and/or expanded by the media and methods described herein may include (but are not limited to) one or more of the following: macrophages, NK cells, helper T cells, regulatory T cells, cytotoxic T cells, inhibitory T cells, B cells, cancer cells, smooth muscle cells, cancer stem cells, endothelial cells, tumour-associated endothelial cells, cancer associated fibroblasts, tissue-specific cells (e.g. goblet cell and/or enterocyte for colorectal cancer) and/or adipose cells.

Table A, provides the detail of an exemplary medium which supports the culture, maintenance, expansion and/or propagation of the mixed population of cells derived from a sample or biopsy. As stated, a medium of this type shortens the maintenance/expansion period needed to achieve a population of cells sufficient for (bio)printing.

TABLE A
Exemplary expansion Medium formulation

| Reagent | Volume | | Percentage | Concentration | |
|---|---|---|---|---|---|
| DMEM/F12 | 437.5 | mL | 87.5% | N/A | |
| Fetal Hyclone 1 | 50 | mL | 10% | N/A | |
| (Primary Cells) | | | | | |
| MEM Vitamin | 5 | mL | 1% | N/A | |
| Solution 100X | | | | | |
| Penicillin | 5 | mL | 1% | 100.0 | ug/mL |
| Streptomycin | | | | | |
| Insulin, Human | 1250 | uL | 0.25% | 10.0 | ug/mL |
| Recombinant, zinc | | | | | |
| solution | | | | | |
| Hydrocortisone | 1040 | uL | 0.2% | 0.2 | ug/mL |
| bFGF | 40 | uL | 0.008% | 0.004 | ug/mL |
| bEGF | 15 | uL | 0.003% | 0.003 | ug/mL |

As stated, cells maintained and/or expanded using a medium of this disclosure may be mixed with a bioink and prepared for (bio)printing to. Where the cells are derived from a tumour sample or biopsy, the maintained and/or expanded cells may be used in the (bio)printing of tumour constructs using 3D-printing as detailed herein. Quality control of the (bio)printed tumours is performed using viability testing on day 1, 7, 14, as well as cellular composition profiling on day 14 using immunofluorescence techniques to assure a high level of original tissue mimicry.

In cases where, despite media supplements, extended cell culture is required, the cell mixture prior to (bio)printing may be supplemented with differentiated immune cells (as described herein) according to the ratios determined by the initial tumour characterisation.

### DETAILED DESCRIPTION

The disclosure will now be further described, by way of example only, with reference to the following Figures.

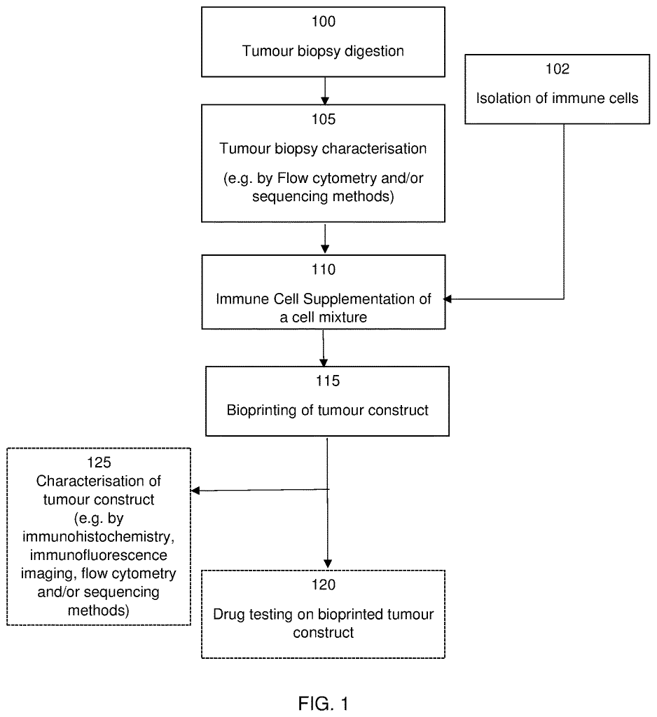

FIG. 1 shows a general overview of an exemplary method of preparing a tumour construct in accordance with an example of the disclosure; and

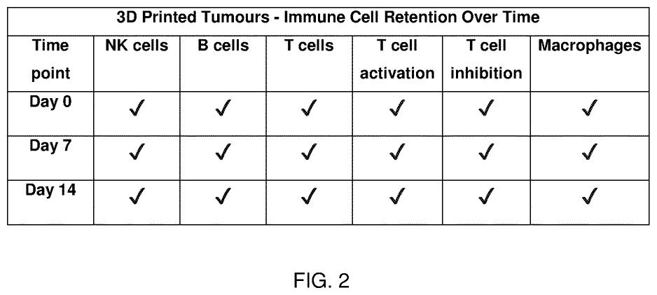

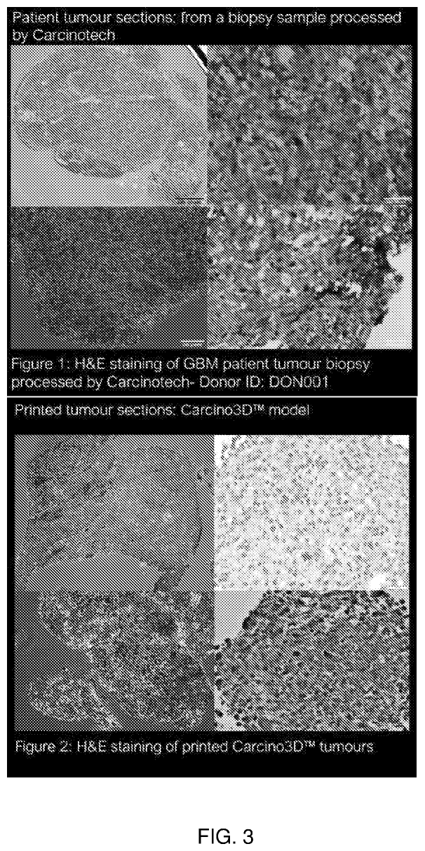

FIG. 2 shows the results of investigations into the immune cell retention in the tumour constructs made in accordance with the methods described herein at day 0, day 7 and day 14 (wherein a tick indicates that the immune cell was present in the construct).

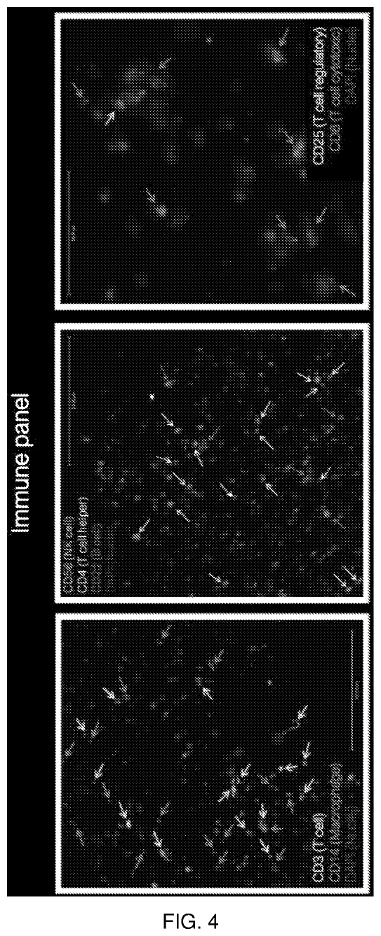

FIG. 3. Histology comparison of glioblastoma (GBM) tumour. The top four images provide H&E-stained patient tumour sections from a biopsy sample obtained from a subject with GBM. The bottom four images provide H&E-stained printed tumour sections using cells obtained from the same patient.

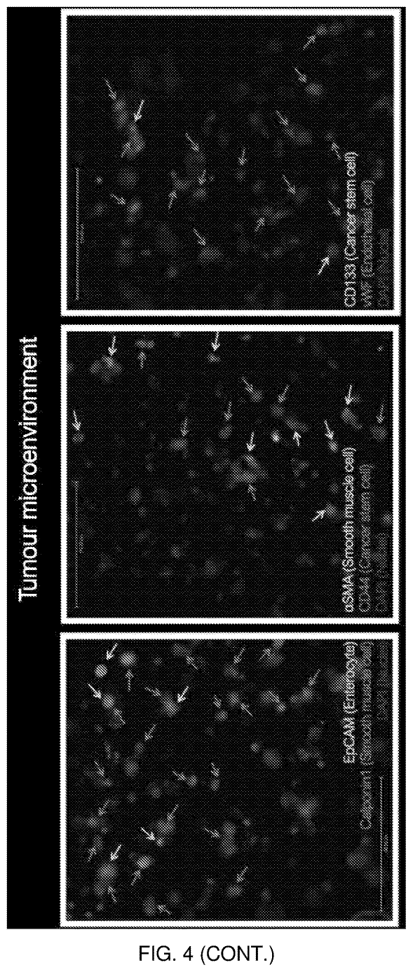

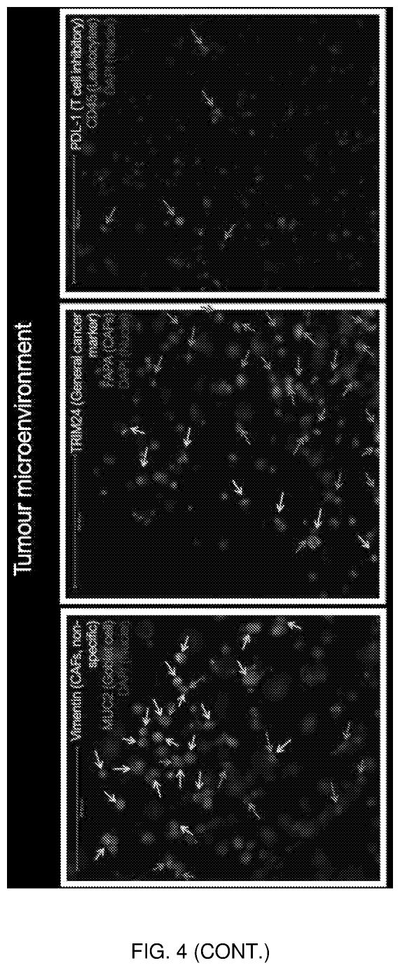

FIG. 4. Characterisation of colorectal tumour model generated by a method of the present disclosure. Immunofluorescence imaging was used to characterise various cell types present in the printed tumour model produced using a biopsy obtained from a colorectal cancer patient.

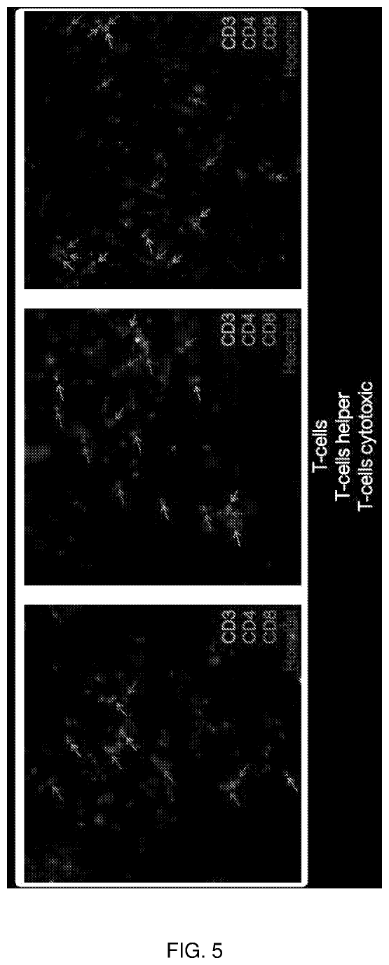

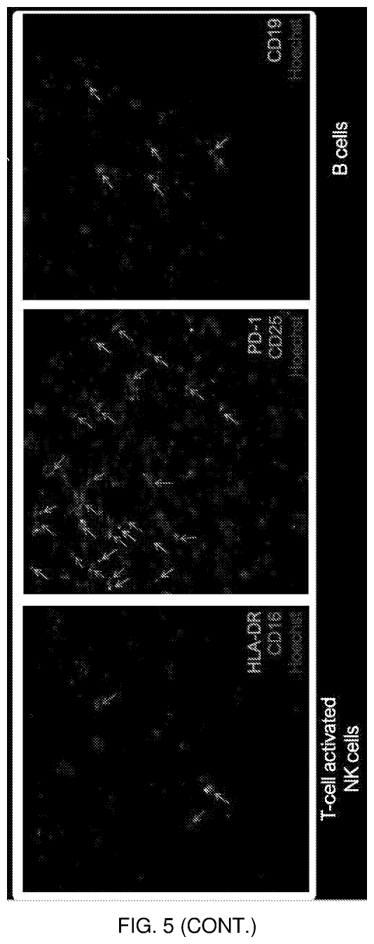

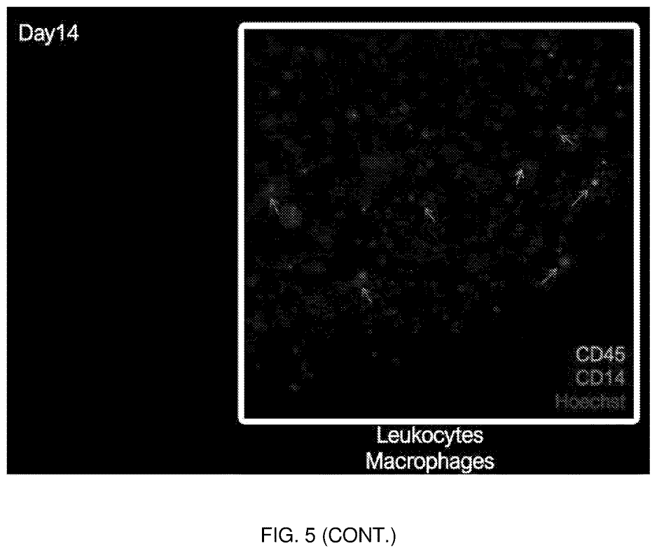

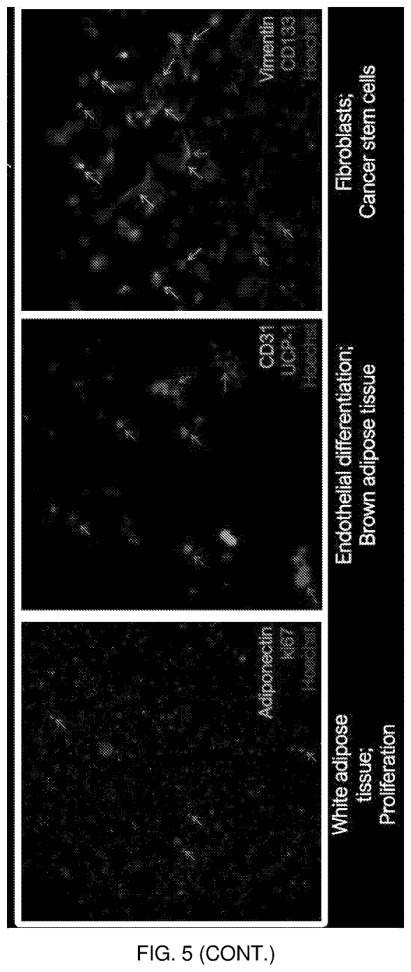

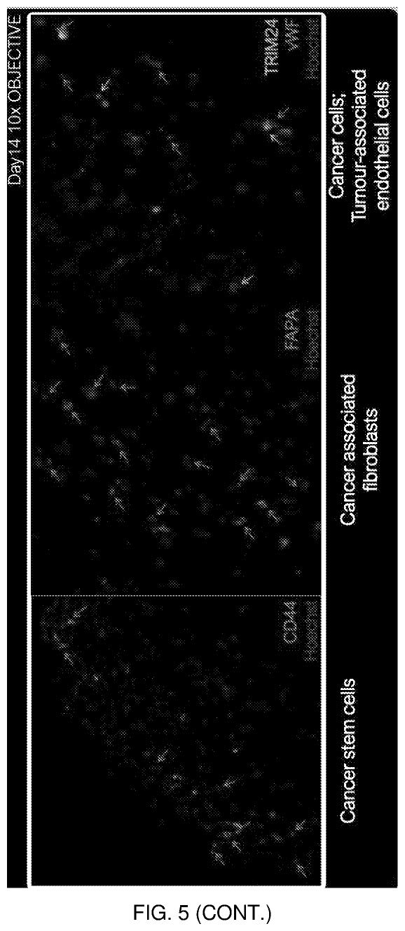

FIG. 5. Characterisation of ovarian tumour model generated by a method of the present disclosure. Immunofluorescence imaging was used to characterise various cell types present in the printed tumour model produced using a biopsy obtained from an ovarian cancer patient.

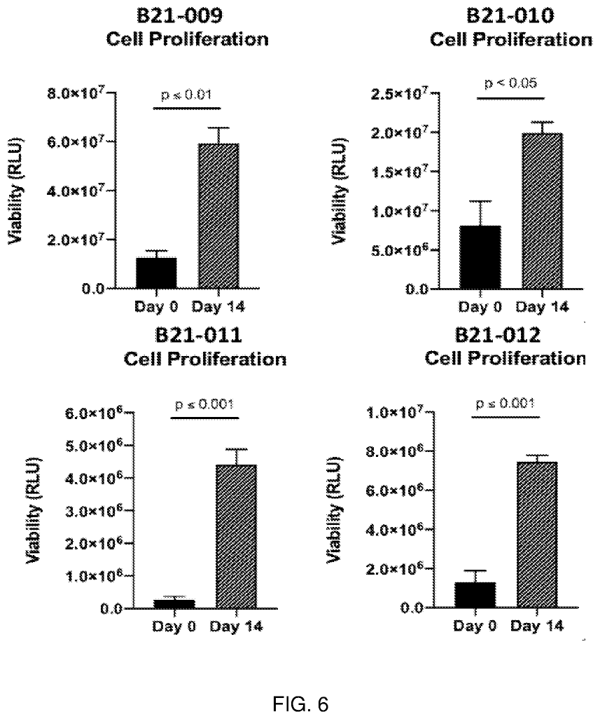

FIG. 6. Cell viability analysis of four (bio)printed constructs using a method detailed herein.

### EXAMPLE METHODS

An exemplary method for preparing a tumour construct is illustrated in FIG. 1. The method comprises step (100) of performing a digestion on a tumour biopsy obtained from a patient, step (102) of isolating immune cells from a patient sample, step (105) of characterising the tumour, step (110) of supplementing a cell mixture with immune cells, and step (115) of (bio)printing the tumour construct. Whilst it is shown as preceding the step of (bio)printing the tumour construct in FIG. 1, in some other examples, the step of supplementing with immune cells may be conducted after the tumour construct has been (bio)printed. In some examples, the method further comprises step (125) characterising the tumour construct and/or step (120) of performing drug testing on the tumour construct.

Each of these steps are described in more detail below.

### Dissociation of Tumour Tissue (an Example of Step 100, FIG. 1)

1. Submerge tissue in room temperature PBS (sample is typically collected in PBS 1× at room temperature).
2. Centrifuge at 100×g at room temperature for 5 min to pellet cells and tissue pieces.
3. Carefully discard supernatant by pipetting and resuspend tissue in 5 mL or more of warm (37° C.) DTC Complete Medium to cover tissue.
   a. Note: For larger tissue (larger than 1 cm³), use multiple rounds of mincing.
   b. Note: Dead cells will not pellet effectively at 100×g and will be contained in the supernatant with other, non-cellular tissue components and secreted factors.
4. Transfer tissue and DTC Complete Medium into a 60 mm petri dish.
5. Mince tissue in experimental medium with scalpel to obtain ~1-3 mm³ pieces.
6. Transfer minced tissue and cells in experimental medium into 15 or 50 ml conical tubes, as dictated by the total volume of the cell and medium suspension.
7. Centrifuge tissue and cells in experimental medium at 100×g at room temperature for 5 min.
8. Discard supernatant by pipetting and add ~4.7 mL of warm experimental medium.
   a. Note: This volume of experimental medium leaves room for ~300 μL of enzyme solutions in the next step and is recommended for tissue that was originally ~1 cm³ in size. For larger tissue, the volumes in Step 8 and Step 9 should be increased proportionately to match tissue size. For example, ~9.4 mL of warm experimental medium would be used in Step 8 for tissue that was originally ~2 cm³ in size.
9. Add 1 mg/mL Collagenase I (prepared in HBSS) and 100 Kunitz/mL DNase I (prepared in dH₂O), and mix with serological pipet.
10. Incubate the tube on a nutating platform (18 rpm) in an incubator (37° C., 5% CO₂) for 30 min (60 min if required).
11. Remove tubes from the incubator and carefully triturate (pipette 25-50 times) the cell suspension using a 10 mL serological pipet. When complete, the cell suspension should look homogeneous and have no visible tissue pieces.
12. Strain with 70 μm cell strainer into a new 50 mL conical tube.
13. Strain flow-through from Step 12 with 40 μm cell strainer into a new 50 ml conical tube.
14. Wash with 10 mL of warm (37° C.) DTC Complete Medium through the 40 μm strainer into the same tube.
15. Centrifuge the collected strained cell suspension at 100×g at room temperature for 10 min, discard supernatant by pipetting.
16. If pellet contains red blood cells or platelets, add 5 mL of ACK lysis buffer following manufacturer protocols, mix with serological pipet, and leave at room temperature for 60 seconds to allow for hypotonic lysis.
17. Add an equal volume of warm DTC Complete Medium (1:1 proportion) to the 50 ml tube, centrifuge at 100×g at room temperature for 10 min, and discard supernatant.
18. Resuspend cells in warm DTC Complete Medium and count cells to quantify viable cells using Trypan Blue.
19. Cells are now ready to be prepared for further analysis/culture.
   a. If flow cytometry analysis is to be performed on a different day, or if the cells need to be preserved for long-term storage, cryopreservation is required. This can be performed per a previously established protocol (Leelatian et al., 2015).

### Isolation of PBMCs (an Example of Step 102, FIG. 1)

Density gradient centrifugation can be used to isolate mononuclear cells from peripheral blood, cord blood, and bone marrow by exploiting differences in density between the various leukocytes and the density gradient medium. Granulocytes and erythrocytes have a higher density than mononuclear cells (MNCs) and therefore sediment through the density gradient medium layer during centrifugation. To isolate mononuclear cells from peripheral blood, cord blood, and bone marrow, it is recommended to use a medium with a density of 1.077 g/mL, such as Lymphoprep™ or Ficoll-Paque™.
i. Ensure all materials are all at room temperature before proceeding (15-25° C.)
ii. Add 15 mL of density gradient medium to the SepMate™ tube by carefully pipetting it through the central hole of the SepMate™ insert.
iii. Dilute sample with an equal volume of PBS+2% FBS, and mix gently.
iv. Keeping the SepMate™ tube vertical, add the diluted sample by pipetting it carefully down the sidewall of the tube. The sample will mix with the density gradient medium above the insert.
v. Centrifuge at 1200×g for 10 minutes at room temperature, with the brake on.
vi. Pour off the top layer, which contains the enriched MNCs, into a new tube, perform in one swift motion.
vii. Wash enriched MNCs with PBS+2% FBS, centrifuging at 300×g for 8 minutes at room temperature. Repeat this step.
viii. Perform a cell count using a small volume of enriched PBMC suspension (≤50 uL) mixed with trypan blue (1:1), read using Countess to attain viability percentage.
ix. Record total cell count and viability.

### SepMate™-15

SepMate™-15 is designed to process 0.5-5 mL of initial sample.

A minimum packed RBC volume of 0.25 mL is required. For samples with low hematocrits, the minimum sample volume may therefore be greater than 0.5 mL.

There is a maximum packed RBC volume of 3 mL. For samples with very high hematocrits, the maximum sample volume may therefore be less than 5 mL.

### SepMate™-50

SepMate™-50 is designed to process 4-17 mL of initial sample.

A minimum packed RBC volume of 2 mL is required. For samples with low hematocrits, the minimum sample volume may therefore be greater than 4 mL.

There is a maximum packed RBC volume of 12 mL. For samples with very high hematocrits, the maximum sample volume may therefore be less than 17 mL.

Differentiation of PBMC-Derived Monocytes into NK, Macrophage, and T Cells

As a result of cell culture, immune cell population present in grown DTCs (disseminated tumour cells) depletes over time. Therefore, it was found that in order to achieve the most accurate representation of primary tumour tissue, immune cell composition requires replenishing before (bio)printing (bio)printing of 3D models. Collecting blood samples from subjects or patients alongside the tumour tissue (“matched blood samples”) allows for isolation of peripheral blood mononuclear cells (PBMC) that can be differentiated into NK, macrophage, and T cells, and used to restore heterogenicity of tumour tissue.

### Phase 1

### Macrophage Isolation and Differentiation Protocol

1. Count and analyze the isolated PBMCs for monocyte content, (e.g. using the FSC/SSC plot of a flow cytometer)
2. Prepare complete RPMI 1640 medium by supplementing RPMI 1640 medium with FBS to a final concentration of 10%, 2 mM L-glutamine (if using medium not currently supplemented with GlutaMAX). Bring medium to 37° C. Optional: Supplement media with 1% penicillin-streptomycin (5,000 units/mL).
3. Resuspend cell concentration to 2×10⁶ cells/mL in complete RPMI 1640 medium.
4. Transfer resuspended cell solution to a cell culture dish.
5. Incubate the culture dish for 24 hours in 5% CO₂ incubator at 37° C. to allow the monocytes to adhere to dish.
6. In a sterile conical tube, prepare complete RPMI 1640 medium with M-CSF Recombinant Human Protein at a final concentration of 40-50 ng/mL. Optional: Add 20 ng/ml IL-4 Recombinant Human Protein.
7. Replace media in culture dish with the prepared media containing M-CSF and IL-4 (if using).
8. Incubate cells for 6 days in 5% CO₂ incubator at 37° C. Within the 6 days, replenish media with new complete RPMI 1640 medium supplemented with 40-50 ng/ml of M-CSF Recombinant Human Protein (Optional; and 20 ng/ml IL-4 Recombinant Human Protein) every 3-4 days. Check under microscope for cell health and confluence.
9. Cells are ready to harvest when cells exhibit more granules in the cytoplasm and are a bit elongated. In addition, cells should be more adherent to the culture plate. When cells are ready to harvest, discard old media, and rinse dish twice with 1×PBS, discarding PBS after each rinse.
10. Add 10 mL 10 mM EDTA to each culture dish, and let sit for 10 minutes, or until cells dissociate from dish at room temperature.
11. Collect cells in a 50 ml conical tube and centrifuge at 300-400×g for 4-5 minutes at room temperature.
12. Discard the supernatant and rinse cells with 1×PBS.
13. Centrifuge cells at 300-400×g for 4-5 minutes.
14. Discard the supernatant and resuspend cells in flow cytometry staining buffer or desired medium.
15. Maintenance of the culture.

### Isolation of NK and T-Cells Using Immunomagnetic Negative Selection

NK and T-cells were isolated using EasySep™ Human Cell isolation protocol (negative selection) (obtained from StemCell Technologies, Inc.).

In brief, EasySep™ Isolation Cocktail was added to the whole blood sample. The mixture was incubated and then EasySep™ RapidSpheres™ were added to the cell suspension. The mixture was incubated and then the tube was placed into the EasySep™ magnet. The magnet and tube were then inverted to pour the enriched cell suspension into a new tube. RapidSpheres™ were then added to the new tube containing the enriched cells. The tube was then removed from the magnet and incubated for a second separation. The magnet and tube were then inverted and the enriched cell suspension was poured into a new tube. The isolated cells were then ready to use.

NK differentiation

### Day 0

1. Add 500 μL of ImmunoCult™ NK Cell Expansion Coating Material (1×) per well of a non-tissue culture-treated 24-well plate.
2. Incubate at room temperature (15-25° C.) for 2 hours.
3. Aspirate coating material from the 24-well plate. Rinse the well with D-PBS. Aspirate D-PBS just prior to use.
4. Prepare ImmunoCult™ NK Cell Expansion Medium:
5. Thaw ImmunoCult™ NK Cell Expansion Supplement at room temperature (15-
6. 25° C.) or overnight at 2-8° C. Mix thoroughly.
7. Add 100 μL of ImmunoCult™ NK Cell Expansion Supplement to 9.9 mL of
8. ImmunoCult™ NK Cell Base Medium. Mix thoroughly.
   NOTE: If not used immediately, store ImmunoCult™ NK Cell Expansion Medium at 2-8° C. for up to 4 weeks.
9. Perform a viable cell count using Trypan Blue.
10. Add 5×10\hat{ }5 NK cells or PBMCs to 500 μL of ImmunoCult™ NK Cell Expansion Medium (1×10\hat{ }6 cells/mL).
11. Add 500 μL of cell suspension (prepared in step 7) to one coated well of the 24-well plate. Incubate at 37° C. and 5% CO₂ for 3 or 4 days.

### Day 3 or 4

12. Carefully add 500 μL of ImmunoCult™ NK Cell Expansion Medium per well of the 24-well plate. Incubate at 37° C. and 5% CO₂ for 3 or 4 days.

### Day 7: Harvest and Reseed

13. Prepare a new 24-well plate as described in steps 1-3.
14. Gently pipette up and down in the well to ensure all cells are in suspension, then transfer cell suspension to an appropriate tube. Rinse the wells with 1 mL DPBS and transfer to tube.
15. Centrifuge tube at 300×g for 10 minutes. Aspirate the supernatant and resuspend the cell pellet in ImmunoCult™ NK Cell Expansion Medium. Perform a viable cell count.
16. Add 2×10⁵ cells to 1 mL of ImmunoCult™ NK Cell Expansion Medium (2×10⁵ cells/mL). These instructions are for a 24-well plate; if using alternative cultureware, refer to the Table.
17. Add 1 mL of cell suspension (prepared in step 14) to one coated well of the 24-well plate prepared in step 10.
18. Incubate at 37° C. and 5% CO₂ for 3 or 4 days.

### Day 10 OR 11: Harvest and Reseed

19. Prepare a new 24-well plate as described in steps 1-3, then harvest and reseed cells as described in steps 11-15. Incubate at 37° C. and 5% CO₂ for 3 or 4 days.

### Day 14

20. Gently pipette cells up and down to ensure all cells are in suspension. Transfer cells to an appropriate tube. These expanded cells are ready for assays or analysis as required. For some donors, cell cultures can be extended beyond 14 days for greater expansion of NK cells.

### Optional Extended 21-Day Culture

21. Prepare a new 24-well plate as described in steps 1-3, then reseed cells as described in steps 11-15. Incubate at 37° C. and 5% CO₂ for 3 or 4 days.

### Day 17 Or 18: Harvest and Reseed.

22. Prepare a new 24-well plate as described in steps 1-3, then harvest and reseed cells as described in steps 11-15. Incubate at 37° C. and 5% CO₂ for 3 or 4 days.

### Day 21

23. Gently pipette cells up and down to ensure all cells are in suspension. Transfer cells to an appropriate tube. These expanded cells are ready for assays or analysis as required.

### T Cells

### Day 0

1. Prepare fresh complete ImmunoCult™-XF T Cell Expansion Medium as follows: Add IL-2 to ImmunoCult™-XF T Cell Expansion Medium. Mix thoroughly.
   NOTE: Complete ImmunoCult™-XF T Cell Expansion Medium must be prepared fresh on each day of use.
2. Seed viable human T cells (isolated with EasySep™ Human T Cell Isolation Kit) in fresh complete ImmunoCult™-XF T Cell Expansion Medium (prepared in step 2a) at 1×10⁶ cells/mL.
3. To activate T cells, add 25 μL/mL of ImmunoCult™ Human CD3/CD28/CD2 T Cell Activator (Catalog #10970) or ImmunoCult™ Human CD3/CD28 T Cell Activator (Catalog #10971) to the cell suspension. Incubate cells at 37° C. and 5% CO₂ for 3 days.

### Day 3

4. Mix the cell suspension thoroughly and perform a viable cell count. Increase the volume of the cell suspension 8-fold (adjust the viable cell density to −1.0-2.5×10⁵ cells/mL) by adding fresh complete ImmunoCult™-XF T Cell Expansion Medium. Incubate at 37° C. and 5% CO₂ for 2 days.

### Day 5

5. Mix the cell suspension thoroughly and perform a viable cell count. Increase the volume at least 4-fold (adjust the viable cell density to −1.0-3.0×10⁵ cells/mL) by adding fresh complete ImmunoCult™-XF T Cell Expansion Medium. Incubate at 37° C. and 5% CO₂ for 2 days.

### Day 7

6. Mix the cell suspension thoroughly and perform a viable cell count. Increase the volume at least 4-fold (adjust the viable cell density to −1.0-6.0×10⁵ cells/mL) by adding fresh complete ImmunoCult™-XF T Cell Expansion Medium. Incubate at 37° C. and 5% CO₂ for 3 days.

### Day 10

7. Harvest cells if the desired cell number is achieved.
8. OPTIONAL: Perform a viable cell count and maintain cell density at 0.5-1.0×10⁶ cells/mL by adding fresh complete ImmunoCult™-XF T Cell Expansion Medium. Incubate at 37° C. and 5% CO₂ for 2 days, then harvest cells.
9. For longer-term expansion (>12 days) of human T cells:
   a. Harvest and resuspend the expanded T cells at 1×10\hat{ }6 cells/mL in fresh complete ImmunoCult™-XF T Cell Expansion Medium.
   Restimulate by adding 25 μL/mL of ImmunoCult™ Human CD3/CD28/CD2 T Cell Activator or ImmunoCult™ Human CD3/CD28 T Cell Activator.
   b. Incubate at 37° C. and 5% CO₂. Every 2-3 days adjust cell density by adding fresh complete ImmunoCult™-XF T Cell Expansion Medium.
   NOTE: Ensure to add fresh complete medium every 2-3 days; do not wait more than 3 days between medium additions.

### Phase 2—Testing Differentiation Efficiency

Any known methods in the art may be employed to test differentiation efficiency, such as using flow cytometry. By way of example only, the following antibodies may be used for flow cytometry analysis.

| | | Cell |
|---|---|---|
| Cells | Antibodies used | characteristics |
| NK | CD3, CD56 | CD3− CD56+ |
| T helper | CD3, CD4 | CD3+ CD4+ |
| T cytotoxic | CD3, CD8 | CD3+ CD8+ |
| Macrophages | CD14, CD45 | CD14+ CD45+ |

After confirmation of surface marker expression, immune cells can be added to the cultured dissociated tumour cells directly before (bio)printing.

### Characterisation of the Patient Sample (an Example of Step 105, FIG. 1)

### Flow Cytometry

1. Harvest, wash the cells (single cell suspension) and adjust cell number to a concentration of 1-5×10⁶ cells/ml in ice cold FACS Buffer (PBS, 10% FBS). Cells are usually stained in polystyrene round-bottom tubes. However, they can be stained in any container for which you have an appropriate centrifuge e.g. test tubes, Eppendorf tubes, and 96-well round-bottomed microtiter plates. It is always useful to check the viability of the cells which should be around 95% but not less than 90%.
2. Add 100 μl of cell suspension to each tube.
3. Add 0.1-10 μg/ml of the primary labelled antibody (check manufacturer's protocols for appropriate concentrations). Dilutions, if necessary, should be made in FACS buffer.
4. Incubate for at least 30 min at room temperature or 4° C. in the dark. This step will require optimization.
5. Wash the cells 2 times by centrifugation at 1500 rpm for 5 minutes and resuspend them in 200 μl of ice cold FACS buffer. Keep the cells in the dark on ice or at 4° C. in a fridge until analysis.
6. If you use primary unlabelled antibody after completing step 5 do the following: Dilute the fluorochrome-labelled secondary antibody in FACS buffer at the optimal dilution (according to the manufacturer's instructions), resuspend cells in this solution and incubate for at least 20-30 minutes at room temperature or 4° C. in the dark. Wash the cells 2 times by centrifugation at 1500 rpm for 5 minutes and resuspend them in 200 μl of ice cold FACS buffer. Keep the cells in the dark on ice or at 4° C. in a fridge until analysis.
7. If you need to preserve cells for several days or are analysing human, infectious materials, or bacteria, after completing step 5 instead of resuspending cells in 200 μl FACS buffer, add 100 μl 1-4% paraformaldehyde and incubate for 10-15 min at room temperature. Centrifuge your samples at 1500 rpm for 5 min and resuspend them in 200 μl ice cold PBS. Fixation will inactivate most biohazardous agents, minimize deterioration, and help to maintain the integrity of your samples. The amount of fixative needed for different sample types will require optimization by the user.
8. Analysis: for best results, analyse the cells on the flow cytometer as soon as possible. For extended storage (16 hr) as well as for greater flexibility in planning time on the cytometer, resuspend cells in 1-4% paraformaldehyde to prevent deterioration.
9. If you use viability dye, please refer to the manufacturer's protocol.
10. For compensation use compensation beads or samples stained with only one antibody conjugated to a fluorophore.
11. Flow cytometry machines, such as MaxQuant 10, may be used for analysis of the samples. Alternatively, or in addition, the sample may be characterised using known sequencing methods in the art, such as RNA-seq, whole exome sequencing and/or whole-genome sequencing. The sequencing method may comprise single cell sequencing and/or bulk sequencing.

### Cell Mixture Printing (an Example of Step 115, FIG. 1)

1. Warm up a Cellink Lamnink cartridge to room temperature. By way of example only, the table below provides materials for the cell mixture printing process:

| Material: | Supplier: |
|---|---|
| Cellink Laminink | Cellink |
| Clear cartridges 3 cc | Cellink |
| Sterile conical (bio)printing nozzles | Cellink |
| BioX or Inkredible series bioprinter | Cellink |
| Well plate or petri dish | Corning |
| Crosslinking agent (inc. with bioink) | Cellink |
| Cells and cell culture medium | Various |
| 3 ml syringes with luer lock connections | Cellink |
| Female/female luer lock adaptor or | Cellink |
| CELLMIXER | |

2. Mix ten-parts bioink (1 mL) with one-part cell suspension (100 μL).
   a. Resuspend 11 million cells in 100 μL cell culture medium.
   b. Transfer the 100 μL cell suspension to a 3 ml syringe using a female/female luer lock adaptor.
   c. Transfer 1 mL of bioink to a 3 ml syringe using a female/female luer lock adaptor.
   d. Attach the bioink syringe to the syringe with cell suspension by pushing the bioink back and forth between the syringes.
   e. Transfer the cell containing bioink back to the cartridge and cap it
3. Place the room tempered CELLINK Laminink in the printhead and cap with a printing nozzle of choice. The recommended nozzle size is 22 G.
4. Identify the (bio)printing parameters from the table below:

TABLE 1
Recommended minimal extrusion pressure* (±2 kPa) used for
printing continuous filaments at 21-25° C. ^{with cells}/without
cells. Again, ‘with cells’ assumes a mixture of one part
cell suspension to ten parts bioink. For information about filament
diameter, see Printing parameters in the Application note. For
highly concentrated cell suspensions, the pressure needs to
be increased towards the pressure used for undiluted bioink.

Printing speed (mm/sec)

| Nozzle size (G) | 5 | 10 | 15 | 20 |
|---|---|---|---|---|
| 22 | 4/ [symbol] | 5/8 | 5/9 | [symbol] /11 |
| 25 | 5/8 | 6/10 | [symbol] /11 | [symbol] /12 |
| 27 | 6/10 | 8/14 | 9/ [symbol] | 11/1 [symbol] |

*Note this is only a recommended reference of starting pressures. The actual pressure needed will vary depending on the preparation procedures (amount of bioink and actual temperature of the bioink) as well as the fitting of the piston in the cartridge and the leveling of the print surface. This table was generated with a printhead temperature at 22° C. for ‘without cells’ and 27° C. ‘with cells’ and with a bioink dilution with a low concentration of cells. [symbol]
indicates data missing or illegible when filed

5. Set the parameters, place the well plate or petri dish on the print bed and run the print.
6. Crosslink the structure using the CaCl₂ crosslinking solution by submerging the cell-laden constructs in 30-50 μL of the crosslinking solution for 30 sec-5 min depending on the construct size (typically 1 min will be sufficient). Remove crosslinking solution and rinse constructs with basal culture media once (80-100 μL).
7. After crosslinking and washing, add the desired medium to the constructs and place in incubator. Incubate the constructs in cell culture medium in standard culture conditions (37° C., 5% CO₂ and 95% relative humidity) or according to your application.

### Spheroid Printing (an Example of Step 115, FIG. 1)

By way of example, a protocol of printing spheroids is also provided herein. Spheroid (bio)printing methods may be employed to achieve a more efficient method to develop patient-derived models. Multicellular spheroids can be used as functional units to create larger tissue structures, making them highly representative, with validation of these models via biomarker expression for each respective cancer, and drug screening against previously approved cancer therapeutics to ensure model reproducibility and accuracy. The details disclosed herein provide complex models which better recapitulate the tumour microenvironment.
1. Pre-form multicellular spheroids within low-adhesion, rounded-bottom well plates following in-house SOPs, using a 200,000+ cells/mL seeding density. (Adjust seeding density according to model requirements, in particular taking into account the tumour size).
2. Spheroid formation is observed in culture for 7 days, with viability measurement prior to 3D (bio)printing.
3. Harvest cell spheroids and use gravity sedimentation to perform washes. (We intend to use CELLINK's Bio CellX bioprinter which will give optimal experimental conditions for spheroid mixing and dispensing, providing increased model reproducibility.)
   a) Resuspend spheroids in fresh culture media and transfer to the Bio CellX in appropriate cartridge, along with customized bioink for respective cancer model for printing.
   b) Or manually mix spheroid suspension with selected bioink for dispensing via CELLINK's BioX6.
4. Spheroid-laden bioink is bioprinted into well plates for further culture of constructs. Incubate contructs after cross-linking of bioink, leave for 24-48 hrs before performing a media change.
5. Perform characterisation of cancer assembloid constructs post-bioprint.
6. The resulting spheroids may be used as multicellular cancer spheroid assembloid models for improved initial viability and maintained cell morphology during culture. These highly innovative constructs have the potential to accelerate drug development, with methods that provide the flexibility to change bioink selection with additional tissue types, whilst reducing the need for animal testing.

### Droplet Printing (an Example of Step 115, FIG. 1)

By way of example only, CELLINK Bio X printing may be used to 3D print biological materials. It can be used for printing small droplets of cells suspended in bio-ink, a mixture consisting of proteins that make up the extra-cellular matrix (ECM). Within the droplets, the cells gradually form 3D spheroids over a period of culture. This produces a good model of the cancer environment as it includes cell-cell interactions, cell-ECM interactions, 3D structure and multiple cell types.

The Bio X is a printer can accommodate different printing methods. For example, pneumatic printing involves extruding a material using air pressure and is good for printing structures and patterns. Printing using a syringe printhead involves mechanically applied pressure to a syringe plunger.

### Supplementation of Bioprinted Tumours (an Example of Step 110, FIG. 1)

Human Peripheral Blood Mononuclear Cell (PBMC) are known to have a short shelf-life in vitro (5-7 days) 2D culture, their survival in the 3D model is not well documented. According to Orrego et al. (December 2018), the presence of tumour infiltrating lymphocytes in a glioma varied from 12 to 21% (17% on average) by flow cytometry analysing tumour dissociated cells.

In order to build an immunologic-based therapy model where immune cells can be harnessed or implemented into the bioprint to attack and kill cancer cells, different types of PBMC survival within the 3D bioprinted tumours need to be monitored.

### Phase 1—Initial Culture.

1. Patient PBMCs are thawed and cultured for 48 h to allow them to recover.

### Phase 2—Day 0 Setup and Testing

2. Different PBMC populations (monocytes, dendritic cells, T cells, B cells, NK cell) are assessed by flow cytometry.
3. The PBMC populations are stained with: Anti-CD3 (T cells), CD56 (NK cells), CD19 or CD20 (B cells), CD 14 or CD16 (macrophages).
4. 1-2 column of bioprints (16 wells) are harvested into PFA after (bio)printing to constitute the baseline (FFPE block for Day 0).

### Phase 3—Day 0 Setup and Testing

5. Then the same number of columns are harvested every week i.e. Day 7, 14, 21 and 28 to constitute FFPE blocks.
6. All blocks are cut and stained with fluorochrome conjugated antibodies to target original PBMC subpopulations.

### Phase 4—Analysis

7. The analysis is carried in 3 different slides (beginning, middle as possible and end) per block to make a statistic valuation of PBMC survival at each time point. Alternatively, this can be assessed by Flow Cytometry after 3D printed tumour digestion.
8. These results are marked against the patient benchmark and repeat runs may be required to fine tune the final ratios

### Using Cell Sorter to Provide Targeted Supplementation of Immune Cells to Bioprinted Tumours (an Example of Step 110, FIG. 1)

By way of example, this section provides an approach of using a cell sorter to supplement the depleting immune cell population in the primary cell cultures over time and maintain an accurate cell population that is representative of the tumour in situ.

Sterile immune cells are isolated from matched human peripheral blood mononuclear cells (PBMCs) via fluorescence activated cell sorting (FACS) on the day of (bio)printing, so that they can be supplemented into the primary culture as required. Immune cell populations are isolated from PBMCs based on the expression profile shown below using a cell sorter:

| Cell Type: | Marker Expression: |
|---|---|
| T Cells | CD3⁺ |
| T Cells (Helper) | CD3⁺/CD4⁺ |
| T Cells (Cytotoxic) | CD3⁺/CD8⁺ |
| T Cells (Regulatory) | CD3⁺/CD25⁺ |
| T Cells (Activated) | CD3⁺/HLA-DR⁺ |
| T Cell (Inhibitory) | CD3⁺/CD274(PDL-1)⁺ |
| Leucocytes | CD45⁺ |
| NK Cells | CD16⁺ |
| B Cells | CD19⁺/CD22⁺ |
| Macrophages | CD14⁺ |

### Analysis—Day 7, Day 14

Bioprinted tumours are digested using Cell Collect A and the immune cell composition measured via flow cytometry using the antibody panel described in the table above. This determines the immune cell depletion over time (7 or 14 days from printing), and once determined, the immune cell population can be adjusted prior to printing to ensure the immune cell population remains the same at the desired testing day (Day 7 or Day 14) as it was at the point of printing. The immune cell population will be adjusted using the cells isolated from PBMCs via a cell sorter as described above.

RNA Extraction from Bioprinted Tumours (an Example of Step 125)

The integration of cells within the bioink makes the RNA extraction complex and required optimisation of the procedure. In the objective to demonstrate the safety of the (bio)printing technology i.e. not inducing (further) mutation or expression changes of bioprinted cancer cells, the inventors have put in place the sequencing process comparing (by RNA alignment) RNA of original tissue vs. the same tissue after (bio)printing.

Bioprints: Collect at least 48 bioprints, dissociate with Cell Collect A and freeze on dry ice. After thawing disrupt the lysate in the appropriate volume of Buffer RLT (see Table 1). Centrifuge the lysate for 3 min at maximum speed. Carefully remove the supernatant by pipetting and use it in step 2.
2. Add 1 volume of 70% ethanol to the lysate, and mix well by pipetting. Do not centrifuge. Proceed immediately to step 3.
3. Transfer up to 700 μl of the sample, including any precipitate, to an RNeasy Mini spin column placed in a 2 ml collection tube (supplied). Close the lid, and centrifuge for 15 s at ≥8000×g. Discard the flow-through.

### DNase Digestion:

If using the RNase-Free DNase Set for the first time, prepare DNase I stock solution by injecting 550 μl RNase-free water into the DNase I vial using an RNase-free needle and syringe. Mix gently by inverting the vial.
For long-term storage of DNase I stock solution, divide it into single-use aliquots and store at −20° C. for up to 9 months. Thawed aliquots can be stored at 2-8° C. for up to 6 weeks.
4. Add 350 μl Buffer RW1 to RNeasy column, close lid, centrifuge for 15 s at ≥8000×g (≥10,000 rpm). Discard flow-through.
5. Add 10 μl DNase I stock solution to 70 μl Buffer RDD. Mix by gently inverting the tube. Centrifuge briefly.
6. Add DNase I incubation mix (80 μl) directly to RNeasy column membrane, and place on benchtop (20-30° C.) for 15 min.
7. Add 350 μl Buffer RW1 to RNeasy column, close lid, centrifuge for 15 s at ≥8000×g. Discard flow-through.
8. Add 700 μl Buffer RW1 to the RNeasy spin column. Close the lid, and centrifuge for 15 s at ≥28000×g. Discard the flow-through.
9. Add 500 μl Buffer RPE to the RNeasy spin column. Close the lid, and centrifuge for 15 s at ≥8000×g. Discard the flow-through.
10. Add 500 μl Buffer RPE to the RNeasy spin column. Close the lid, and centrifuge for 2 min at ≥8000×g.

Optional: Place the RNeasy spin column in a new 2 ml collection tube (supplied). Centrifuge at full speed for 1 min to dry the membrane.
11. Place the RNeasy spin column in a new 1.5 ml collection tube (supplied). Add 30-50 μl RNase-free water directly to the spin column membrane. Close the lid, and centrifuge for 1 min at ≥8000×g to elute the RNA.
12. If the expected RNA yield is >30 μg, repeat step 7 using another 30-50 μl of RNase-free water, or using the eluate from step 11 (if high RNA concentration is required). Reuse the collection tube from step 11.
   DNA Extraction from Cells and 3D Bioprinted Tumours (an Example of Step 125, FIG. 1)
1. Harvest at least 24 bioprints and place them into 1.5 ml Eppendorf tube containing 1 ml of Cell Collect A.
2. Place the samples on a shaking platform for 30 min or until fully dissolved—it is recommended to shake at 4° C. to slow cell signalling pathways but can also be done at room temperature or 37° C. You may need to aspirate the solution with a pipette to fully dissociate the bio-ink
3. Place the cell strainer over a centrifuge tube and wet with sterile PBS
4. Filter the cell suspension and then centrifuge at 400 g for 3-4 minutes to collect cell pellet.
5. Discard the supernatant and add 180 ul of Qiagen Alt lysis buffer add 20 μl Proteinase K. Mix thoroughly by vertexing, and incubate at 56° C. until the tissue is completely lysed. Vortex occasionally during incubation to disperse the sample or place in a thermomixer, shaking water bath or on a rocking platform. Lysis time varies depending on the type of tissue processed. Lysis is usually complete in 1-3 h.
6. Vortex for 15 s. Add 200 μl Buffer AL to the sample, and mix thoroughly by vortexing. Then add 200 μl ethanol (96-100%), and mix again thoroughly by vortexing. Note: A white precipitate may form on addition of Buffer AL and ethanol.
7. Pipet the mixture into the DNeasy Mini spin column placed in a 2 ml collection tube. Centrifuge at ≥6000×g (8000 rpm) for 1 min. Discard flow-through and collection tube.
8. Place the DNeasy Mini spin column in a new 2 ml collection tube (provided), add 500 μl Buffer AW1, and centrifuge for 1 min at ≥6000×g (8000 rpm). Discard flow-through and collection tube.
9. Place the DNeasy Mini spin column in a new 2 ml collection tube, add 500 μl Buffer AW2, and centrifuge for 3 min at 20,000×g (14,000 rpm) to dry the DNeasy membrane. Discard flow-through and collection tube.
10. Following the centrifugation step, remove the DNeasy Mini spin column carefully so that the column does not come into contact with the flow-through, since this will result in carryover of ethanol. If carryover of ethanol occurs, empty the collection tube, then reuse it in another centrifugation for 1 min at 20,000×g (14,000 rpm).
11. Place the DNeasy Mini spin column in a clean 1.5 ml or 2 ml microcentrifuge tube, and pipet 200 μl Buffer AE directly onto the DNeasy membrane. Incubate at room temperature for 1 min, and then centrifuge for 1 min at ≥6000×g (8000 rpm) to elute. Elution with 100 μl (instead of 200 μl) increases the final DNA concentration in the eluate, but also decreases the overall DNA yield.
12. For maximum DNA yield, repeat elution once as described in step 10. This step leads to increased overall DNA yield. A new microcentrifuge tube can be used for the second elution step to prevent dilution of the first eluate. Alternatively, to combine the eluates, the microcentrifuge tube from step 10 can be reused for the second elution step. Note: Do not elute more than 200 μl into a 1.5 ml microcentrifuge tube because the DNeasy Mini spin column will come into contact with the eluate.

### Characterisation of Cryosections: Immunofluorescent Staining

The following is a general procedure guide for preparation and staining of cryosections. Depending to the fluorochrome attached to the antibody, the target antigen, the detection microscope (fluorescent or confocal) and the quality of cryosections the user must determine optimal conditions for each antigen of interest.

### Tissue Preparation for Cryosectioning (Steps 1-9):

1. Remove the tumorsphere or bioprint from the plate using tweezers and place it directly in a pre-labelled tissue base mould.
2. Cover the entire the tumorsphere or bioprint with cryo-embedding media (e.g. OCT).
3. Place the base mould containing the tumorsphere or bioprint on top of dry-ice and leave it to be frozen completely.
4. Store the frozen tissue block at −80° C. until ready for sectioning.
5. Transfer the frozen tissue block to a cryotome cryostat (e.g. −20° C.) prior to sectioning and allow the temperature of the frozen tissue block to equilibrate to the temperature of the cryotome cryostat.
6. Section the frozen tissue block into a desired thickness (typically 4-10 μm) using a cryostat.
7. Place the tissue sections onto glass slides suitable for immunohistochemistry (e.g. Superfrost).
8. Dry the tissue sections for 1-2 h or overnight at room temperature.
9. Fix the slides in pre-cooled methanol (100%) at −20° C. for 10 minutes and then wash 3× with PBS. Sections can then be stored in a sealed slide box at −80° C. for later use.
   Immunofluorescence Staining of Frozen Tissue Sections with Conjugated-Primary Antibodies (Steps 10-16):
1. Incubate slides with 1× animal-free blocking buffer for 30 mins at 4° C. (dilute the buffer to 1× using ddH₂O). For slides with multi-sections, use a hydrophobic pen (liquid blocker) to draw a circle around the section to contain the buffer on top of the section (avoid the section to dry out).
2. Prepare fluorochrome-conjugated antibodies in the 1× buffer while blocking (50-100 μL per section).
3. Discard the blocking buffer and apply 50-100 μl of the appropriately diluted primary antibody (in antibody dilution buffer) on top of each section. Cut parafilm and delicately place it on top the antibody-drop, make sure that the antibody solution did not overflow the liquid blocker circle around the section and incubate overnight at 4° C. (for thicker sections (≥10 mm) and PFA-fixed slides, you may wish to consider triton-100 permeabilization or antigen retrieval before antibody staining). The incubation must be carried in a humidified chamber (damp Kimtech paper tissue in the incubation chamber to protect from liquid evaporation). It is good practice to include, at least, one fluorescent isotype control in each staining. From this step, slides must be protected from light to avoid fluorochrome bleaching.
4. After the incubation delicately remove the parafilm and then wash the slides in PBS-triton (0.1%)—Tween 20 (0.5%) for 5 min twice, and then with PBS alone twice (washing with pre-cooled buffers might help to preserve tissue quality so wash at 4° C.).
5. Discard the washing buffer and drain dry slides by using the Kimtech paper tissue to drain excess buffer.
6. Apply drops of Diamond mounting medium on each slide before delicately placing a coverslip on top of it, make sure all sections on the slides are covered with the mounting medium avoid air bubbles then leave slides to cure for overnight-24 hours protected from light.
7. Store at 4° C. until ready to be imaged.

### Characterisation of Bioprinted Spheroids: Immunofluorescent Staining (an Example of Step 125, FIG. 1)

The following is a general procedure guide for preparation and staining of bioprinted spheroids. Depending to the fluorochrome attached to the antibody, the target antigen, the detection microscope (fluorescent or confocal) and the quality of bioprints the user must determine optimal conditions for each antigen of interest.

### 3D Spheroid Preparation for Staining (Steps 1-4):

1. Washing: use a multi-channel pipette to transfer 200 ml of 1×PBS per well in a black plate (glass bottom for fluorescent imaging). Remove the tumorsphere or bioprint from the plate using tweezers and transfer it to the black imaging plate. Once all of the tumorspheres/bioprints have been transferred, put the plate on an oscillating shaking platform for 10 minutes at room temperature (RT) to wash away any media.
2. Fixation-permeabilization: use a multi-channel pipette to discard PBS from each well and replace it with 100 ul PBS solution containing 4% PFA and 2% triton-X100 and return to the shaking platform for 1 hour at RT.
3. Optional: further permeabilization can be done by incubating with 200 ul PBS-triton-×100 (2%) for 2 hours to maximise spheroid permeabilizing.
4. Washing: remove the fixation-permeabilisation solution using a multichannel pipette and wash the spheroids 2× with 200 ul PBS-triton-X100 (0.2%) for 20 mins at RT on the shaking platform.
5. Blocking: once washed, block the tumorspheres/bioprints in 100 ul PBS containing 0.2% triton-X100 and 5% foetal bovine serum (FBS) for 30 mins at RT on the shaking platform.
   Immunofluorescence Staining of Frozen Tissue Sections with Conjugated-Primary Antibodies (Steps 5-11):
1. Primary antibody staining: replace the blocking buffer with 100 ml of your desired primary antibody diluted in PBS containing 0.2% triton-X100 and 5% FBS. Incubate overnight in a cold room (4° C.) on a shaking platform.
2. Washing: remove the antibody solution using a multichannel pipette and wash the spheroids 3× with 200 ml PBS containing 0.2% triton-X100 and 0.1% Tween20 for 15 mins at RT on the shaking platform.

### IF THE PRIMARY ANTIBODY IS FLUOROPHORE-CONJUGATED, PROCEED TO STEP 9. IF NOT, CONTINUE THE PROTOCOL FROM STEP 7.

3. Secondary antibody staining: replace the wash buffer with 100 ml of your desired secondary antibody diluted in PBS containing 0.2% triton-X100 and 5% FBS. Incubate for 90 mins at RT on the shaking platform.
4. Washing: remove the antibody solution using a multichannel pipette and wash the spheroids 3× with 200 ml PBS containing 0.2% triton-X100 and 0.1% Tween20 for 15 mins at RT on the shaking platform.
5. Counterstaining: incubate the tumorspheres/bioprints in 5 μM Hoechst 33342 for 30 mins at RT on the shaking platform.
6. Washing: remove the counterstain solution using a multichannel pipette and wash the spheroids 3× with 200 ml PBS containing 0.2% triton-X100 and 0.1% Tween20 for 15 mins at RT on the shaking platform. Once complete, wash once more with PBS only for 15 mins at RT using the shaking platform.
7. Storage and Preservation: remove the PBS using a multichannel pipette and add 50 μl of 4% PFA in PBS, wrap the plate in tinfoil to omit light and store at 4° C. until ready to be imaged.

### Bioprinted Tumour Digestion for Endpoint Characterisation

Following the (bio)printing of 3D models and subsequent cell growth, it may be preferable to extract single cells from bioprints for downstream quantification at viability specific timepoints of interest. To recover cells digestion of the bioink is required using both manual and enzymatic methods to dissolve the surrounding ECM and achieve a single-cell suspension. The retrieved cells are then used to further characterise the tumour models post-(bio)printing to ensure the correct cellular composition and tumour microenvironment is reflective of the original tumour biopsy and maintained throughout culture. A multi-stage method can be used to digest the two main components of the bioink, alginate and nanofibrillated cellulose:
1. Harvested bioprints are placed into sodium citrate buffer (50-55 mM) at 37° C. for 3-5 min with gentle agitation. This stage will breakdown the alginate component of the bioink.
2. Filter digested cell suspension through a 70 um Cell Strainer followed by centrifugation at 200 g for 5 min.
3. After removal of the supernatant, replace with cellulase solution (100-300 μg/mg) at 37° C. for 4-5 hr with manual agitation.
4. Repeat filtration step as above.
5. Resuspend cells in trypsin-EDTA for 2-3 min to dissociate cell aggregates.
6. To inhibit trypsin activity resuspend in serum-containing culture media.
7. Finally centrifuge suspension at 200 g for 5 min to achieve a pure cell pellet ready for cell counts and endpoint characterisation.

### Culturing, Freezing and Thawing of 3D Bioprinted Constructs

Without wishing to be bound by theory, 3D bioprinted constructs need to be fed on a regular basis, and can also be frozen and kept at −150° C. for long term storage and dry ice shipping. The present disclosure provides an exemplary protocol for maintenance, cryopreservation, and thawing of 3D bioprinted cancer models.

### Culturing

Always monitor the bioprints for signs of infection, such as medium turbidity and rapid medium colour change. Observe under the microscope. Appropriate aseptic technique must always be used when handling plates with bioprinted constructs.
Feed bioprinted constructs three times a week (e.g. Mon, Wed, Fri) with the 3D Culture Medium (see: 4. Materials Required). Monitor the colour change of the medium and increase feeding frequency if required.
To avoid damaging the bioprints, do not remove the whole supernatant when feeding (e.g., remove 150 μl of old medium, leaving 50 μl, and add 150 μl of fresh medium).

### Freezing & Thawing

Plates can be frozen at day 7. After thawing, bioprinted constructs is typically cultured for additional 7 days before drug treatments. Protocol below is for a 96-well plate. For other plate formats adjust the media volumes accordingly. Appropriate aseptic technique must be used throughout the cryopreservation and thawing process.

### Freezing

1. Remove the culture medium from the wells.
2. Add 100 μl of cold freezing medium (Cryo SFM) to each well.
3. Seal the plate with plate-sealing film.
4. Wrap the plate in aluminium foil and transfer into the styrofoam box.
5. Tape and label the box (including an indication where the top of the plate is) and transfer it to the −80° C.
6. Fill in the Cryopreservation of Cell Lines form (FRM/TC/003).
7. After 16-24 hours at −80° C., transfer the plate to −150° C. (transport on dry ice) for long term storage.
8. Frozen plate is shipped on dry ice. Upon receipt it can either be stored at −150° C. or thawed (see section below for protocol)

### Thawing

1. Warm up the culture medium to 37° C.
2. Remove the frozen plate from −150° C. freezer and place on dry ice for the time of transport.
3. Under sterile conditions, add 100 μl of warm culture medium to frozen wells.
4. Place the plate in a 37° C. bead bath** until thawed (it should take around 5-10 min).
5. Remove the whole medium from the wells, careful not to aspirate the bioprinted constructs.
6. Add 200 μl of warm culture medium and place the plate in the incubator (37° C., 5% CO₂).
7. Fill in the Thaw of Cells form (FRM/TC/001)
8. Change the media the next day, and feed at three times a week onwards.
** Do not use a water bath to thaw plates.

### Drug Testing Using Tumour Constructs—Viability Assay (an Example of Step 120, FIG. 1)

### 2D Cell Culture Viability Assessment

1. Seed cells in a white 96-well tissue culture plate.
2. Perform desired treatment.
3. Equilibrate the plate at RT for 30 min. Equilibrate thawed CellTiter-Glo reagent at RT for 30 min also.
4. Mix CellTiter-Glo gently to ensure homogenous solution.
5. To each well, add a volume of CellTiter-Glo equal to the volume of cell culture medium (e.g., 50 μl+50 μl)
6. Include background control: medium+CellTiter-Glo
7. Place on an orbital shaker for 2 min, 100 rpm, RT.
8. Incubate for further 10 min at RT to stabilise the luminescence signal. Incubate in the dark, preferably in the GloMax plate holder, to avoid the phosphorescence of the white plate.
9. Record luminescence: GloMax, Luminescence Quick Read, integration time 0.3 seconds.

### 3D Cell Culture Viability Assessment: Tumospheres and Hydrogel Constructs→No Hydrogel Digestion

1. Perform desired treatment.
2. Transfer the constructs to a white assay plate containing 100 μl of fresh medium (alternatively: 1×PBS).
3. Equilibrate the plate at RT for 30 min. Equilibrate thawed CellTiter-Glo reagent at RT for 30 min also.
4. Mix CellTiter-Glo gently to ensure homogenous solution.
5. To each well, add a volume of CellTiter-Glo equal to the volume of cell culture medium/PBS (e.g., 100 μl+100 μl).
6. Include background control: medium/PBS+CellTiter-Glo
7. Place on an orbital shaker for 5 min, 100 rpm, at RT.
8. Incubate for further 25 min at RT to stabilise the luminescence signal. Incubate in the dark, preferably in the GloMax plate holder, to avoid phosphorescence of the white plate.
9. Record luminescence: GloMax, Luminescence Quick Read, integration time 0.3 seconds.

### 3D Cell Culture Viability Assessment: Hydrogel Constucts→Hydrogel Digestion

1. Perform desired treatment.
2. Equilibrate Cell Collect A at RT for 30 min
3. Transfer the constructs to a white assay plate containing 100 μl of appropriate bioink digestion solution (e.g., Cell Collect A, enzyme-based reagent that digests alginate)
4. Place the plate on an orbital shaker for 30 min, 60 rpm, at RT.
5. In the meantime: equilibrate thawed CellTiter-Glo reagent at RT for 30 min also.
6. After incubation, pipet the well contents up and down (×10) to help dissociate the bioink *.
7. Mix CellTiter-Glo gently to ensure homogenous solution.
8. To each well, add a volume of CellTiter-Glo equal to the volume of the bioink digestion solution (e.g., 100 μl+100 μl).
9. Include background control: bioink digestion solution+CellTiter-Glo.
10. Place on an orbital shaker for 5 min, 100 rpm, at RT.
11. Incubate for further 25 min at RT to stabilise the luminescence signal. Incubate in the dark, preferably in the GloMax plate holder, to avoid phosphorescence of the white plate.
12. Record luminescence: GloMax, Luminescence Quick Read, integration time 0.3 seconds.
* The hydrogel will most likely not dissociate completely, and it is ok. The bioink digestion solution will loosen its structure anyway, and thus facilitate permeation of CellTiter-Glo reagent into the construct.

### Drug Testing Using Tumour Constructs—Cytotoxicity Assay (an Example of Step 120, FIG. 1)

### Reagent Reconstitution and Storage

This procedure requires at least 15 minutes equilibration time.

1. AK detection reagent (AKDR) •Add 10 ml (5 plate kit) or 20 ml (10 plate kit) of assay buffer to the vial containing the lyophilized AK detection reagent. •Replace the blue screw cap and mix gently. •Allow the reagent to equilibrate for 15 minutes at room temperature.

Use reconstituted reagent within 6 hours, or 24 hours if stored at 2° C.-8° C. Unused reagent can be aliquoted into polypropylene tubes and stored at −20° C. for up to two months. Once thawed, reagent must not be refrozen and the reagents should be allowed to reach room temperature before use, without the aid of artificial heat.

Protocol 1: Adherent/Suspension Cells. Cells Cultured in Luminescence Compatible Plate; 96 Well Format
1. Bring all reagents up to room temperature before use.
2. Reconstitute the AK detection reagent (AKDR) in assay buffer (see pg 2). Leave for 15 minutes at room temperature to ensure complete rehydration.
3. Remove the culture plate from the incubator and allow it to cool to room temperature for at least 5 minutes.
4. Program the luminometer to take an immediate 1 second integrated reading of appropriate wells.
5. Add 100 μl of AKDR to each well and wait 5 minutes.
6. Place plate in luminometer and initiate the program.
   Protocol 2: Adherent/Suspension Cells. Supernatant Sampling Procedure. Cells Cultured in Luminescence Incompatible Plate; 96 Well Format
1. Bring all reagents up to room temperature before use.
2. Reconstitute the AK detection reagent (AKDR) in assay buffer.
3. Leave for 15 minutes at room temperature to ensure complete rehydration.
4. Remove the culture plate from the incubator and allow it to cool to room temperature for at least 5 minutes.
5. Program the luminometer to take an immediate 1 second integrated reading of appropriate wells.
6. Transfer 20 μl of cell supernatant to a luminescence compatible 96 well plate.
7. Add 100 μl of AKDR to each well and wait 5 minutes.
8. Place plate in luminometer and initiate the program.
   Protocol 3: Adherent/Suspension Cells. Cells Cultured in Luminescence Compatible Plate; 384 Well Format
1. Bring all reagents up to room temperature before use.
2. Reconstitute the AK detection reagent (AKDR) in assay buffer. Leave for 15 minutes at room temperature to ensure complete rehydration.
3. Remove the culture plate from the incubator and allow it to cool to room temperature for at least 5 minutes.
4. Program the luminometer to take an immediate 1 second integrated reading of appropriate wells.
5. Add 25 μl of AKDR to each well and wait 5 minutes.
6. Place plate in luminometer and initiate the program.
   Protocol 4: Adherent/Suspension Cells. Supernatant Sampling Procedure. Cells Cultured in Luminescence Incompatible Plate; 384 Well Format
1. Bring all reagents up to room temperature before use.
2. Reconstitute the AK detection reagent (AKDR) in assay buffer. Leave for 15 minutes at room temperature to ensure complete rehydration.
3. Remove the culture plate from the incubator and allow it to cool to room temperature for at least 5 minutes.
4. Program the luminometer to take an immediate 1 second integrated reading of appropriate wells.
5. Transfer 5 μl of cell supernatant to a luminescence compatible 384 well plate.
6. Add 25 μl of AKDR to each well and wait 5 minutes.
7. Place plate in luminometer and initiate the program.

### Drug Testing Using Tumour Constructs—Apoptosis Assay—CellTox-Green Cytotoxicity (an Example of Step 120, FIG. 1)

### Endpoint Measurement Protocol (2D & 3D)

1. Perform desired treatment.
2. Make sure to include the wells without test compounds for both negative (untreated) and positive (cytotoxicity) controls, as well as empty wells with medium blanks.
3. Incubate for the desired exposure period.
4. Reagent preparation (on the day of the assay):
   Thaw CellTox Green assay components (Dye, Assay Buffer, and Lysis Solution) at 37° C. Vortex to ensure homogeneity. Centrifuge briefly if necessary.
   Prepare the required volume of 2× CellTox Green dye in the Assay Buffer; 100 μl per well is needed (e.g., 2 μl of the dye per 1 mL of Assay Buffer). Protect from light!
5. If needed, remove an appropriate volume of the exposure medium from the assay plate so that only 100 μl per well is left.
6. Cytotoxicity control (positive control):
   30 minutes prior to reading, add Lysis Buffer to the cells assigned for positive control at a 1:25 ratio (4 μl per 100 μl of culture medium in a well).
7. To perform the assay, add 100 μl of 2× CellTox Green dye per well.
8. Mix by orbital shaking (120 rpm*) for 1 minute to ensure homogeneity.
9. Incubate the plate for 15 minutes at room temperature, protected from light.
10. Optional: before measuring fluorescence, mix again by orbital shaking for 1 minute.
11. While protecting from light, transfer the plate to GloMax and choose the relevant setting (96 well with or without lid).
12. Measure the fluorescence at excitation/emission wavelengths 485-500/520-530 nm (on GloMax Discover use the following filter: ex/em 475/500-550 nm)

### Kinetic Study Protocol (“Express, No-Step Addition Method”: 2D & 3D)

1. Prepare the cells (2D cultures, 3D tumorspheres, or 3D bioprints) in a 96-well plate at least a day before the treatment.
2. Make sure to include the wells without test compounds for both negative (untreated) and positive (cytotoxicity) controls*, as well as empty wells with medium blanks.
3. Reagent preparation (on the day of the assay):
   Thaw CellTox Green assay components (Dye and Lysis Solution) at 37° C. Vortex to ensure homogeneity. Centrifuge briefly if necessary.
   Prepare the required volume of 2× CellTox Green dye in the cell culture medium; 100 μl** per well is needed (e.g., 2 μl of the dye per 1 mL of medium). Protect from light!
4. Remove the medium from the wells and replace it with 100 μl** of 2× CellTox Green dye dilution.
5. Perform desired treatment. Add 100 μl* of prepared 2× compound dilutions to the wells containing cells in 2× CellTox Green dye.
6. Incubate the cells for up to 72 hours and take measurements at chosen time-points.
7. Cytotoxicity control (positive control):
   30 minutes prior to reading, add Lysis Buffer to the cells assigned for positive control at a 1:25 ratio (8 μl per 200 μl of culture medium in a well).
   Required: include the positive control at the last time-point. Optional: include the positive control at all time points.
8. Optional: before measuring fluorescence, mix the plate by orbital shaking for 1 minute. Protect from light.
9. Transfer the plate to GloMax (protecting from light) and choose the relevant setting (96 well with lid).
10. Measure the fluorescence at excitation/emission wavelengths 485-500/520-530 nm. Return the plate to the incubator after reading.
*If you want to include a positive control at all time-points (optional), you need separate wells for each time-point. This is because positive control cells need to be lysed 30 min prior to the reading. The positive control can also be done at the last time-point only.
** For smaller cell numbers in 2D cultures, up to 10,000 cells/well, 50 μl, instead of 100 μl, can be used (final reaction volume=100 μl). For bioprints, stick to 100 μl.
   Multiplexing with CellTiter-Glo Viability Assay

For more details on CellTiter-Glo Viability Assay see SOP/AM/004. Assay can be performed in either white or black plate. Clear plates are not acceptable for luminescence measurements.
1. After the final CellTox Green measurement, equilibrate the plate to room temperature. If needed, remove the excess exposure medium, so that 100 μl (for bioprints and tumorspheres >10,000 cells/well), or 50 μl (for 2D cultures or tumorspheres≤10,000 cells/well) of medium per well is left.
2. Thaw the CellTiter Glo reagent and equilibrate it to room temperature.
3. Add the equal volume of CellTiter-Glo to each well (50 μl or 100 μl, 1:1 CellTiter-Glo: medium ratio).
4. Incubate* the plate at room temperature:
   2D cultures: place on the orbital shaker for 2 minutes (120 rpm), then incubate for further 10 min.
   3D cultures: place on the orbital shaker for 5 minutes (120 rpm), then incubate for further 25 min.
5. Record luminescence: GloMax, Luminescence Quick Read, integration time 0.3 s. Choose a relevant 96 well setting (with or w/o lid).
* If a white plate is used, shield it from light to avoid phosphorescence. Incubation can be done in the GloMax plate holder.

### Drug Testing Using Tumour Constructs—Apoptosis Assay—Caspase-Glo 3/7 Reagent Preparation and Storage (an Example of Step 120, FIG. 1)

1. Equilibrate the Caspase-Glo® 3/7 Buffer and lyophilized Caspase-Glo® 3/7 Substrate to room temperature before use.
2. Transfer the contents of the Caspase-Glo® 3/7 Buffer bottle into the amber bottle containing Caspase-Glo® 3/7 Substrate. Mix by swirling or inverting the contents until the substrate is thoroughly dissolved to form the Caspase-Glo® 3/7 Reagent. Buffer volumes are 2.5 ml for G8090, 10 ml for G8091 and G8093, and 100 ml for G8092.

Storage: The reconstituted Caspase-Glo® 3/7 Reagent may be stored at 4° C. for up to 3 days with no loss of activity compared to that of freshly prepared reagent. Reconstituted reagent stored at 4° C. for 1 week will give a signal approximately 90% of that obtained with freshly prepared reagent, while reconstituted reagent stored at 4° C. for 4 weeks will give a signal approximately 75% of that obtained with freshly prepared reagent. Reconstituted reagent that has been refrozen and stored at −20° C. for 1 week will give a signal approximately 75% of that of freshly prepared reagent, and refrozen reagent stored at −20° C. for 4 weeks will give a signal approximately 60% of that of freshly prepared reagent.

### Standard Protocol for Cells Cultured in a 96-Well Plate

1. Before starting the assay, prepare the Caspase-Glo® 3/7 Reagent. Allow the reagent to equilibrate to room temperature. Mix well.
2. Remove 96-well plates containing treated cells from the incubator and allow plates to equilibrate to room temperature.
3. Add 100 μl of Caspase-Glo® 3/7 Reagent to each well of a white-walled 96-well plate containing 100 μl of blank, negative control cells or treated cells in culture medium. Because of the sensitivity of this assay, be careful not to touch pipet tips to the wells containing samples to avoid cross-contamination. Cover the plate with a plate sealer or lid.
Note: If you are reusing pipet tips, do not touch pipet tips to the wells containing samples to avoid cross contamination.
4. Gently mix contents of wells using a plate shaker at 300-500 rpm for 30 seconds. Incubate at room temperature for 30 minutes to 3 hours, depending upon the cell culture system. The optimal incubation period should be determined empirically.
Note: Temperature fluctuations will affect the luminescence reading. If the room temperature fluctuates, use a constant-temperature incubator.
5. Measure the luminescence of each sample in a plate-reading luminometer as directed by the luminometer manufacturer.
Note: Directions are given for performing the assay in a total volume of 200 μl using 96-well plates. However, the assay can be adapted to other volumes, provided the 1:1 ratio of Caspase-Glo® 3/7 Reagent volume to sample volume is used (e.g., 25 μl of sample and 25 μl Caspase-Glo® 3/7 Reagent in a 384-well format).

### Drug Testing Using Tumour Constructs—Hypoxia Assay (an Example of Step 120, FIG. 1)

1. Dissolve Image-iT hypoxia reagent powder in DMSO (1.4 mL) to make stock solution (1 mM).
Note: This stock solution can be stored at −20° C. for 6 months or at 4° C. for one week.
2. Dilute the stock solution in a cell culture medium to make a final concentration 1-10 μM.
3. Transfer cells/constructs to appropriate plate or petri dish for imaging.
4. Cover with Image-iT red working solution and incubate at 37° C. for 30-60 minutes*
5. Replace working solution with fresh cell culture medium.
5. Incubate the cells at 37 C for 2-4 hours* under preferred oxygen conditions.
6. Image cells using filers suitable for ex./em. 490/610 nm.
*Larger 3D constructs will need more than 2D cultures.

### Drug Testing Using Tumour Constructs→Cytokine Array (an Example of Step 120, FIG. 1)

### Reagent Preparation

These are general reagent preparation guidelines. If possible, all reagents should be prepared fresh, directly before use. Keep all reagents on ice during preparation. Reagents should only be used in their 1× working concentration.

### Storage:

Once thawed, store array membranes and 1× Blocking Buffer at ≤−20° C., and all other component at 4° C.
1× Wash Buffers can be stored at 4° C. for up to 1 month.
Protease Inhibitor Cocktail stock solution can be stored at −20° C. for up to 12 weeks.

### Lysis Buffer Preparation:

To prepare 1× Lysis Buffer, dilute 2× Lysis Buffer with ultrapure water.
To prepare 7× Protease Inhibitor Cocktail (PIC) stock: dissolve one tablet of complete EDTA-free PIC in 1.5 mL of ultrapure water or 1×PBS.
Prepare the Lysis Buffer by adding 143 ul of 7×PIC to 857 ul of 1× Lysis Buffer.

### Other Buffer Dilutions:

Wash Buffers I and II are supplied as 20× concentration. Dilute each Wash Buffer 20-fold with distilled or deionized water.
Biotin-Conjugated Anti-Cytokines are supplied at 2000× concentration as a small liquid bead. 1 vial is enough to test 2 membranes. Briefly centrifuge each vial prior to reconstitution as the concentrated liquid bead can adhere to the inside walls and cap during transit. Reconstitute by pipetting 2 ml of 1× Blocking Buffer into the 2,000× Biotin-Conjugated Anti-Cytokines vial to prepare the 1× working concentration.
HRP-Conjugated Streptavidin is supplied at a 1000× stock concentration. Mix the 1000×HRP-Conjugated Streptavidin vial well before use as precipitants may form during storage. Dilute 1000×HRP-Conjugated Streptavidin 1000-fold with 1× Blocking Buffer to prepare the 1× working concentration.
Blocking Buffer and Detection Buffers C & D are supplied as 1× working concentration.

### Sample Preparation

The array is compatible with a range of sample types, such as culture media, cell lysates, tissue homogenates, blood, etc. See manufacturer's protocol for details.

1 mL of sample per membrane is needed.

### Culture Medium

No need for dilution. Aspirate the medium, aliquot and freeze.
Medium should be free of recombinant cytokines and growth factors, like EGF! If it is necessary to have them, use uncultured media with additives as a blank.
The kit should be compatible with serum-containing media. Nevertheless, test uncultured media as a sample “blank” to assess baseline signal responses.

### Bioprints→Cell Lysate

1. Culture bioprints in a medium without growth factors.
2. Process constructs made of CELLINK alginate-based bioinks according to SOP/BM/005. All steps are performed on ice:
   a) To digest the bioink, prepare a tube with cold Cell Collect A (10× bioink volume×number of constructs) and transfer constructs there.
   b) Place the tube on the shaker for 30 min.
   c) Facilitate the bioprint disintegration using a pipette.
   d) Place a cell strainer over a centrifuge tube and wet the bottom of the strainer with sterile PBS.
   e) Strain the prepared ECM-cell suspension. Passing through a cell strainer doesn't help with separating the bioink unfortunately, but it can help disintegrate the spheroids. Press the syringe plunger against the strain surface and facilitate spheroid dissociation using circular motions. Rinse the strainer with small volume of sterile PBS.
   f) Centrifuge at 400×g for 3-4 min (cell suspension can also be transferred to a microcentrifuge tube before centrifugation, so it's easier to recover a small number of cells). A cell/bioink pellet will form. Remove the supernatant.
3. Prepare cell lysate:
   a) Resuspend the pellet in ______ ml of 1× Lysis Buffer supplemented with 1×PIC. Recommended lysate concentration is 1×10⁷ cells per 1 mL of Lysis Buffer.
   b) Pipet up and down to facilitate cell lysis.
   c) Incubate on ice with gentle agitation, for 30 min.
   d) Centrifuge at 10,000×g for 10 min at 4° C. to clear the lysate.
   e) Transfer cleared lysate to a fresh tube. Aliquot if possible, and store at −20° C.
4. Measure the protein concentration in the sample using the BCA method (compatible with detergents, see: SOP/AM/xx).
   a) Minimum recommended total protein concentration in the lysate (prior to sample dilution) is 1.0 μg/ul
5. Cell lysate sample must be diluted 5× to 10× with 1× Blocking Buffer, up to the volume of 1 mL (sample volume needed for one array membrane).
   a) For the first experiment, use 200-250 μg of total protein in 1 ml of final (diluted) sample for each array membrane. Optimal amounts of total lysate protein may range from 50-1000 μg per array membrane.

### Assay Protocol

All incubations are performed under gentle agitation (rocking/rotation). Wet or dry, grasp membranes by the edges using forceps. Do not allow membranes to dry out during experiments. Do not pipet the buffers directly onto the membranes, but to the corner of the well instead.

### Day 1

Prepare the reagents/dilutions needed for day 1 and thaw samples on ice. If needed, dilute the samples. Remember to include a background control membrane (medium+FBS).
1. Blocking: incubate membranes with 2 mL 1× Blocking Buffer for 30 min at RT, gentle agitation.
2. Spin samples for 5 min at minutes at 10,000 rpm immediately prior to incubation with the array to remove any particulates that could interfere with detection.
3. Sample: Incubate membranes with 1 mL of prepared sample overnight at 4° C.

### Day 2

Prepare reagents/dilutions needed for day 2.
1. Large Volume Wash: after o/n incubation with the sample, transfer membranes to clean containers and wash with 20 ml of Wash Buffer I for 30 min at RT.
2. Wash I: wash membranes 3 times with 2 mL of 1× Wash Buffer I.
3. Wash II: wash membranes 2 times with 2 mL of 1× Wash Buffer II.
4. Anti-Cytokine Abs: incubate with 1 mL of 1× biotin-conjugated anti-cytokine antibody cocktail for 2 h at RT.
5. Wash I: wash membranes 3 times with 2 mL of 1× Wash Buffer I.
6. Wash II: wash membranes 2 times with 2 mL of 1× Wash Buffer II.
7. HRP-Streptavidin: incubate with 1 mL of 1×HRP-conjugated streptavidin overnight at 4° C.

### Day 3

Prepare reagents/dilutions needed for day 3.
1. Wash I: after o/n incubation with HRP-conjugated streptavidin, wash membranes 3 times with 2 mL of 1× Wash Buffer I.
2. Wash II: wash membranes 2 times with 2 mL of 1× Wash Buffer II.
3. Chemiluminescence detection:
   a) Place membrane onto a sheet of blotting paper, printed side up. Remove excess wash buffer by blotting the membrane edges with another piece of paper.
   b) Transfer the membrane onto the plastic sheet.
   c) Prepare detection buffer by mixing equal volumes of Detection Buffer C and detection Buffer D.
   d) Gently pipette 500 ul of detection mix onto each membrane and incubate for 2 min at RT (no agitation at this step!).
   e) Place another plastic sheet on top of the membrane and press to remove the bubbles (avoid “sliding” the plastic sheet along the membranes' surface).
   f) If using a CCD camera, transfer the sandwiched membranes to the imaging system and expose. If possible, all membranes should be detected at the same time, as this will allow for better direct comparison.
   g) Try multiple exposures to obtain an image with low background and strong positive control signal spots that do not bleed into one another. Typical exposure times are between few seconds to 2 minutes.

To store the membrane, tape the sheets together or wrap in plastic wrap to secure them, and store at ≤−20° C.

### Results

An example 3D bioprinted tumour construct was made using the methods and protocols described above. The immune cell retention of the construct was investigated at day 0, day 7 and day 14. It was confirmed that immune cells (in particular NK cells, B cells, T cells, T cell activation, T cell inhibition, macrophages) were present in the construct at each of these time points. The results are illustrated in FIG. 2.

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

### Results

### Tumour Characterisation

The tumour microenvironment (TME) typically comprises of cells of the immune system (T cells, B cells, dendritic cells, MDSCs, TAMs), a complicated network of fibroblasts, blood vessels, lymphatics and the cells of the cancer itself.

Various techniques, such as histology, flow cytometry, immunohistochemistry were used to investigate cell populations of the tumour constructs produced using the method and culture conditions (DTC Supplemented Medium) of the present disclosure, which are described in detail below.

### I. Histology

### H&E

Haematoxylin & Eosin staining is the staining of tissue sections with Haematoxylin, which stains cell nuclei blue, and Eosin, which stains the extracellular matrix, cytoplasm and other structures varying shades of pink. This allows visualisation of the structure, distribution of cells and morphological changes within a tissue sample.

### MT Chrome

Masson's Trichrome Staining is a histological staining method used for selectively stain collagen, collagen fibers, fibrin, muscles, and erythrocytes. It uses three stains for staining hence the term Trichrome. These are Weigert's Hematoxylin, Biebrich scarlet-acid fuschin solution, and Aniline blue.

By using the tissue processing steps as disclosed herein, it was found the culture conditions of the present disclosure ensured an optimal number of cells can be maintained in culture and used for (bio)printing. A single biopsy sample was able to be printed into 5×96-well plates with one 3D printed tumour construct per well—a total of 480 bioprinted tumours could be produced from a single patient sample. The H&E staining of the patient sample and the printed tumour are similar (FIG. 3).

### II. Immunofluorescence Analysis

Multiplex immunofluorescence allows simultaneous antibody-based detection of multiple markers with a nuclear counterstain on a single tissue section.

Using two exemplary tumour biopsies (a biopsy obtained from a colorectal cancer (FIG. 4) and a biopsy obtained from an ovarian cancer (FIG. 5)), immunofluorescence analysis was used to characterise the cellular components of the tumour biopsies 14 days following (bio)printing.

As shown in FIGS. 4 and 5, immunofluorescence analysis revealed the exemplary bioprinted tumour models generated using the method of the present disclosure maintain all cell types typically found in the original tissue 14 days following (bio)printing.

### III. Viability of Cells of the Tumour Construct

Determining cell viability is extremely important for ensuring bioprinted constructs are proliferating and cells are viable in the constructs. The 3D Cell Viability Assay is designed for determining cell viability in 3D tissues. The assay reagent penetrates large spheroids and has increased lytic capacity—allowing more accurate determination of viability compared to other assay methods. Based on the same chemistry, this 3D assay reagent measures ATP as an indicator of viability, and generates a luminescent readout that is much more sensitive than absorbance or fluorescence-based methods.

Four different tumour construct models generated using the medium and methods disclosed herein were bioprinted (Day 0) and cell viability was assessed 14 days following (bio)printing (Day 14). As exemplified in FIG. 6, the Day 14 results for each model is much higher than Day 0 indicating proliferation, high cell viability and growth. This indicates that the 3D bioprinted models are ready for subsequent analysis or assays, such as drug screening and testing. Compared to existing 3D organoid and spheroid solutions known in the art, the tumour constructs detailed herein are ready for subsequent analysis or assays 14-21 days after (bio)printing, which is a much shorter timeframe compared to other tumour models known in the art, which can take a minimum of 60 days to 90 days to be ready for drug screening.

### Example: Cell Preparation & 3D Bioprinting (BIO X6) (See FIGS. 4-6)

### Introduction and Purpose

Carcinotech GBM model is a multicellular model built progressively by implementing different cancer and supportive cell lines in a ratio that allows cell cohesion and growth in a 3D format before including them in bioink (biogel mimicking animal cell Extracellular Matrix).

Materials Required (with Catalogue Numbers/Suppliers)

| Chemical reagents | Catalogue | Supplier |
|---|---|---|
| Laminink+ | Various | CELLINK |
| 96 well plate | 1005 | ThermoFisher |
| 1 mL sterile syringe | 309628 | BD |
| 3 mL sterile syringe | 309658 | BD |
| Female/female luer lock | OH0000000102 | CELLINK |
| adaptor (connector) | | |
| 3 mL pneumatic cartridge | CSC010300102 | CELLINK |
| Nozzles/needles | Various | CELLINK |

### Cell Harvesting

Donor/cell line harvesting is performed using TrypLE Express Enzyme solution. Cell supernatant is also retained and centrifuged for donor cell cultures, whereas cell-line media can be discarded if no suspended cells are present. Sterile PBS (1× without Ca²⁺/Mg²⁺) is used to wash cultures prior to cell detachment.

Cell counts are conducted to determine their respective concentrations, enabling the calculation of the necessary volumes for the planned bioprinting process.

The number of cells required for the bioprint is calculated by:

N=number wells×number of cells per wells+200 ul more (lost in syringes).

For cell-line models, mix cells proportionately (GBM model example):
50% main cancer cell line (U87MG, U87MG_GFP or DKMG/EGFRvIII, etc.)
28% microglia cell line HMC3
15% astrocyte cell line IHAClone2
5% cancer associated fibroblast cell line (GBMCAF)
2% GBM cancer stem cell (GBMCSC)

At this stage, the cells may be left in the medium and the bioink prepared before moving forward.

### Preparing the Bioink

CELLINK Laminink 411 may be used for bioprinting of our GBM models, the volume of bioprint is calculated based on the previously established ratio:
100,000 cells per 3 ul of ink+100 ul (for waste in the mixing process)

Cells to be combined with the bioink were expanded using a media of this disclosure—in particular a media according to that summarised in Table A (Exemplary expansion Medium formulation) above.

### Centrifuging Cells.

Once the bioink preparation is complete the cell suspension is centrifuged (1500 rpm, 5 min at room temperature in the large-volume centrifuge in Carcinotech CB2 lab).
For practical cell pellet transfer, cell pellet is resuspended in 10 mL complete growth medium (U87MG medium preferably) and transferred into 10 mL Falcon tube.
Repeat the centrifugation as above; discard the supernatant.
Aspirate the cell pellet and transfer into the opening of the luer-connector attached to one of the 1 mL syringes held upright.
Rinse the 10 mL Falcon tube with 10 μL of Hyclone serum using a fine-bore 10 μL pipette tip.
Pool this 10 μL inside the connector with the cell pellet
Adjust the cell pellet to ensure contact with the bioink by gently pulling and pushing the syringe plunger, before connecting it to the other syringe containing the remaining half of the prepared bioink.
Connect both 1 mL syringes:
Mix the cell pellet and bioink to achieve a homogeneous solution by gently pulling and pushing the syringe plungers.
Once a cell homogeneous-bioink solution is obtained, the homogeneous solution is transferred to a 1 ml syringe, emptying the second syringe as much as possible.

### Loading Cell-Bioink

Connect the 3 ml syringe preloaded with CellInk start-ink with the 1 ml syringe containing the homogeneous solution.
Connect the 1 ml syringe containing the homogeneous cell-bioink solution
Load the solution into the 3 ml syringe, emptying the 1 ml syringe

### Bioprinting of 3D Models

Insert well plate for printing
Select new protocol “Droplet” settings:
   SURFACE: choose well plate e.g. 96WP, Corning COSTAR®.
   DROPLET: choose format e.g. single droplet, printhead 1.
      Open well/printhead selection option-select serpentine horizontal.
   PRINTHEADS: choose type e.g. 3 mL pneumatic, 22 G nozzle
   Select printhead settings: Pressure=25-45 kPa range; Extrusion time=0.1 s; Temperature; Extrusion height=0.1 mm.
   PRINTER: enable clean chamber fan, ON
   Select GO TO PRINT
   CALIBRATE PRINTHEADS: Automatic bed levelling—drop surface probe, run, store surface probe away when prompted.
   Automatic calibration—prepared cell laden-bioink cartridge with attached nozzle should be connected to printhead.
Extrude a small volume until bioink flows from cartridge→START PRINT
Monitor printing progress, and adjust printhead settings to influence bioprint size and uniformity.

### Bioprint Crosslinking

Following printing, submerge bioprinted constructs in 100 uL per well of 50 mM CaCl₂ crosslinking agent for 3 mins, then aspirate solution from wells.
Add 100 uL per well of 3D Tumorsphere Complete Media, as a washing step. Then aspirate and discard media.
Finally add 150 uL-180 uL per well of 3D Culture Media, and incubate.

### Expected Outcome/Result (Including Images)

The outcome of this SOP are 3D-printed cell line tumour models in 96-well format.

### Whole-Exome Sequencing and RNA Sequencing Comparison (Tumour Vs Bioprint)

Whole exome sequencing of GBM tumour sample and bioprinted tumours derived from patients DTCs was performed. The analysis revealed that number of mutational changes in the coding regions of tested bioprints was only 0.05% higher than in primary tumour. Gene expression analysis through RNAseq has also been performed. RNA sequencing of GBM tumour sample and bioprinted tumours derived from patients DTCs was performed. Differential expression analysis revealed changes in levels of 376 genes out of >20000 genes, showing a similarity of over 98%.

1. A cell culture medium for expanding cells dissociated from a tumour or tissue sample, said medium comprising:
(a) one or more growth factors; and/or
(b) one or more glucocorticoids;
and optionally,
(c) a serum supplement; and/or
(d) a vitamin supplement; and/or
(e) one or more antibiotics.

2. The cell culture medium of claim 1 comprising insulin, FGF, EGF and hydrocortisone.

3. A method of making a tissue or tumour construct, said method comprising:
(a) determining the cellular profile of a tissue or tumour sample obtained from or provided by a subject;
(b) dissociating the tissue or tumour sample to provide dissociated cells;
(c) expanding the dissociated cells in the presence of the media of any one of claim 1 or 2; and
(d) using the expanded cells to prepare a tissue or tumour construct.

4. A method of making a tumour construct, said method comprising:
(a) determining the cellular profile of a tumour biopsy obtained from or provided by a subject;
(b) preparing a cell mixture to mimic the determined cellular profile of the tumour biopsy;
(c) supplementing said cell mixture with immune cells derived or obtained from, or provided by, said subject; and
(d) using said supplemented cell mixture to prepare a tumour construct.

5. The method of claim 4, wherein the immune cell profile mimics the immune cell profile of the tumour biopsy.

6. The method of any of claims 4 to 5, wherein the immune cells comprise white blood cells, peripheral blood mononuclear cells (PBMCs) or cells derived therefrom.

7. The method of any of claims 4 to 6, wherein the cell mixture comprises single cells and/or spheroids.

8. The method of claim 7, wherein the single cells and/or spheroids are derived from dissociated cells from the tumour biopsy.

9. The method of any of claims 4 to 8, wherein the cell mixture further comprises a bioink and/or one or more growth factors.

10. The method of any of claims 4 to 9, wherein using said supplemented cell mixture comprises depositing said supplemented cell mixture, optionally (bio)printing said supplemented cell mixture.

11. The method of any one of claims 4 to 10, wherein the immune cells are derived or obtained from a blood sample from the same subject from whom the tumour biopsy was obtained or provided.

12. The method of any of claims 4 to 11, wherein the tumour construct is cryopreserved.

13. A construct for mimicking an in vivo tumour environment, the construct comprising:
(a) patient-derived cells from a tumour biopsy obtained from or provided by a subject, wherein said patient-derived cells comprise a cancer cell, a cancer stem cell, a stromal cell, a cancer-associated fibroblast, an endothelial cell, a pericyte and/or an epithelial cell, or a combination thereof; and
(b) immune cells derived from or provided by said subject.

14. The construct of claim 13, wherein the immune cell profile mimics the immune cell profile of the tumour biopsy.

15. The construct of any of claims 13 to 14, wherein the cell composition of the construct is proportionate to the cell composition of the tumour biopsy.

16. The construct of any of claims 13 to 15 further comprising a bioink, wherein the bioink comprises one or more extracellular components.

17. The construct of any of claims 13 to 16, wherein the immune cells comprise white blood cells.

18. The construct of any of claims 13 to 17, wherein the immune cells comprise PBMCs or cells derived therefrom.

19. The construct of any of claims 13 to 18, wherein the immune cells comprise T cells, B cells, NK cells, monocytes, macrophages, and/or dendritic cells, or a combination thereof.

20. The construct of any of claims 13 to 19, wherein the immune cells comprise T cells, NK cells and/or macrophages, or a combination thereof.

21. The construct of any of claims 13 to 20, wherein the cells of the construct are maintained for at least 7 days.

22. The construct of any of claims 13 to 21, wherein the cells of the construct are maintained for at least 14 days.

23. The construct of any of claims 13 to 21, wherein the cells of the construct are maintained for at least 21 days.

24. A method of testing cell response to a treatment, said method comprising:
(a) providing a tumour construct made according to the method of any of claims 3 to 12 or a construct of any of claims 13 to 23;
(b) contacting the tumour construct with the treatment;
(c) optionally, maintaining the tumour construct with the treatment; and
(d) determining the response of one or more cells to the treatment.

25. The method of claim 24, wherein the method optionally further comprises:
determining the cellular profile of one or more cells of the tumour construct prior to and/or subsequent to contacting the tumour construct with the treatment.

26. The method of any of claims 24 to 25, wherein said treatment is selected from: a compound, a drug, an antibody, radiation therapy, ultrasound therapy, radiofrequency therapy, laser therapy, UV therapy, photodynamic therapy, electrochemotherapy, immunotherapy, stem cell therapy, heat therapy, cryotherapy and therapeutic oligonucleotides, or combinations thereof.

27. A bioink formulation for use in a method according to any one of claims 3 to 12, or for use in making a construct according to any one of claims 13 to 23, comprising:
(a) one or more polymers; and/or
(b) one or more extracellular matrix components; and/or
(c) a cell mixture obtained from a tumour biopsy obtained from or provided by a subject; and optionally further comprising:
(d) immune cells obtained from or provided by said subject; and/or
(e) endothelial cells; and/or
(f) one or more growth factors.

28. The bioink formulation for use of claim 27, wherein the cell composition of the cell mixture is proportionate to the cell composition of the tumour biopsy.

29. The bioink formulation for use of any of claims 27 to 28, wherein the immune cell profile mimics the immune cell profile of the tumour biopsy.

30. The bioink formulation for use of any of claims 27 to 29, wherein the cell mixture comprises a cancer cell, a cancer stem cell, a stromal cell, a cancer-associated fibroblast, a pericyte and/or an epithelial cell, or a combination thereof.

31. A bioink formulation for use in a method according to any one of claims 3 to 12, or for use in making a construct according to any one of claims 13 to 23, comprising:
(a) laminin, alginate, collagen, nanofibrillated cellulose and/or fibrinogen, or a combination thereof;
(b) a cell mixture prepared from a tumour biopsy obtained from or provided by a subject; and optionally,
(c) immune cells obtained from said subject.

32. The bioink formulation for use of claim 31, wherein the immune cells are PBMCs or cells derived therefrom.

33. The bioink formulation for use of claim 32, wherein the cell composition of said cell mixture and/or immune cells is proportionate to the cell composition of the tumour biopsy.

34. The bioink formulation for use of claim 33, wherein the immune cell profile mimics the immune cell profile of the tumour biopsy.

35. A kit comprising at least one of each of the following:
(a) a cell mixture prepared from a tumour biopsy obtained from or provided by a subject;
(b) immune cells obtained from said subject; and optionally further comprising:
(c) a culture medium; and/or
(d) instructions for use.

36. The kit of claim 35, further comprising one or more of the following:
(d) one or more extracellular matrix components;
(e) one or more growth factors; and/or
(f) an endothelial cell.

37. The kit of any of claims 35 to 36, wherein the components (a) to (c), and optionally one or more of components (d) to (f), are:
supplied and/or stored separately within the kit; or
are provided in the kit as a bioink formulation.

38. The kit of any of claims 35 to 37, wherein the cell mixture comprises single cells and/or spheroids.

39. The kit of any of claims 35 to 38 wherein the components (a) and (b), and optionally (c), (d) to (f) are cryopreserved.

40. A tumour construct obtainable by the method of any one of claims 3-12.

41. The method of any one of claims 4-12, wherein the step of preparing a cell mixture to mimic the determined cellular profile of the tumour biopsy, comprises dissociating the tumour biopsy and expanding the dissociated cells using the media of claim 1 or 2.

42. A method of making a tissue or a tumour construct, said method comprising:
(a) determining the cellular profile of a tissue or a tumour biopsy obtained from or provided by a subject;
(b) dissociating the biopsy to provide dissociated cells;
(c) expanding the dissociated cells from the biopsy to prepare a cell mixture mimicking the determined cellular profile of the biopsy;
(c) optionally supplementing said cell mixture with immune cells derived or obtained from, or provided by, said subject; and
(d) using said expanded and optionally supplemented cell mixture to prepare a tumour or tissue construct.

43. The method of claim 42, wherein the expanding step uses the medium of claims 1 or 2.
